(19) United States
(12) Patent Application Publication
(10) Pub. No.: US 2026/0248389 A1
(43) Pub. Date: Aug. 27, 2026

(54) MICROENVIRONMENTAL MONITORING UTILIZING DIFFERENT TYPES OF SENSING DEVICES IN A BODY AREA NETWORK ASSOCIATED WITH A SUBJECT

(71) Applicant: LifeLens Technologies, Inc., Ivyland, PA (US)

(72) Inventors: Landy Toth, Doylestown, PA (US); Kevin D’Aquilla, Warminster, PA (US)

(21) Appl. No.: 19/161,430

(22) PCT Filed: Mar. 8, 2024
(86) PCT No.: PCT/US24/19053

§ 371 Date: Sep. 2, 2025

Related U.S. Application Data
(60) Provisional application No. 63/451,773, filed on Mar. 13, 2023.

(51) Int. Cl. A61B 5/00; A61B 5/369; A61B 5/394; A61B 5/398

(52) U.S. Cl. CPC A61B 5/0024; A61B 5/369; A61B 5/394; A61B 5/398; A61B 2560/0242

(57) ABSTRACT

An apparatus comprises a processing device implementing a body area network (BAN) controller for a BAN associated with a subject. The processing device is configured to pair first and second sets of sensing devices with the BAN associated with the subject, the first set of sensing devices configured for physiologic monitoring of the subject and the second set of sensing devices configured for contextual monitoring of an environment of the subject. The processing device is also configured to enable data sharing of physiologic monitoring data obtained from the first set of sensing devices and contextual monitoring data obtained from the second set of sensing devices utilizing the BAN associated with the subject, and to determine, based on the physiologic monitoring data obtained from the first set of sensing devices and the contextual monitoring data obtained from the second set of sseennssiinngg devices, microenvironmental monitoring parameters associated with the subject.

### STATEMENT OF GOVERNMENT RIGHTS

This invention was made with government support under Medical Technology Enterprise Consortium (MTEC) Contract No.: 2019-399 awarded by the Defense Health Agency (DHA). The government has certain rights in the invention.

### TECHNICAL FIELD

The present disclosure relates to the field of networking and, more particularly, to devices and systems for managing networks.

### BACKGROUND

Physiologic monitoring is performed for a range of purposes. Existing technologies, however, are not without shortcomings.

There is a need to measure physiologic parameters of subjects, reliably, simply, and without cables. As the proliferation of mobile and remote medicine increases, simplified and unobtrusive means for monitoring the physiologic parameters of a patient become more important. More reliable, redundant, and user friendly systems are needed that can provide valuable patient data even when operating with limited supervision, expert input, or user manipulation, including in remote locations where power and/or local area networks are not readily available.

### SUMMARY

One illustrative, non-limiting objective of this disclosure is to provide systems, devices, and methods for managing networks, including body area networks including different types of devices. Another illustrative, non-limiting objective is to provide a flexible architecture enabling sharing of contextual and environmental information amount different types of devices that are part of a body area network associated with a subject. Yet another illustrative, non-limiting objective is to provide systems, devices, and methods for physiologic monitoring of subjects, including physiologic monitoring utilizing contextual and environmental information shared amongst different types of devices that are part of a body area network associated with a subject.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

In one embodiment, an apparatus comprises at least one processing device comprising a processor coupled to a memory. The at least one processing device implements a body area network controller for a body area network associated with a subject. The at least one processing device is configured to pair a first set of sensing devices with the body area network associated with the subject, the first set of sensing devices configured for physiologic monitoring of the subject, and to pair a second set of sensing devices with the body area network associated with the subject, the second set of sensing devices configured for contextual monitoring of an environment of the subject. The at least one processing device is also configured to enable data sharing of physiologic monitoring data obtained from the first set of sensing devices and contextual monitoring data obtained from the second set of sensing devices utilizing the body area network associated with the subject, and to determine, based at least in part on the physiologic monitoring data obtained from the first set of sensing devices and the contextual monitoring data obtained from the second set of sensing devices, one or more microenvironmental monitoring parameters associated with the subject.

The contextual monitoring data may comprise information characterizing exposure of the environment of the subject to at least one of: one or more infectious agents; insolation; radiation; blast overpressure; and noise.

At least one of the second set of sensing devices may comprise an electrophysiological measuring device, the electrophysiological measuring device comprising at least one of: an electrooculography sensor; an electroglottography sensor; and an electroencephalography sensor.

At least one of the second set of sensing devices may comprise an optical sensor device, the optical sensor device comprising at least one of: an ambient light sensor; a spectrophotometer sensor; a closed-circuit television sensor; an infrared sensor; and a hyperspectral imager sensor.

At least one of the second set of sensing devices may comprise a rangefinder and mapping device, the rangefinder and mapping device comprising at least one of: a light detection and ranging (LIDAR) sensor; a RADAR sensor; and a miniaturized opto-mechanical sensor.

At least one of the second set of sensing devices may comprise an electrophysiological measuring device, the electrophysiological measuring device comprising at least one of: an electrooculography sensor; an electroglottography sensor; and an electroencephalography sensor.

At least one of the second set of sensing devices may comprise a sensor for sensing at least one of body-exogenous and body-endogenous exposure to one or more biological agents and chemical compounds.

At least one of the second set of sensing devices may comprise a dosimeter configured to evaluate exposure to at least one of blast overpressure, noise and radiation.

At least one of the second set of sensing devices may comprise at least one of: a barometer; an anemometer; an accelerometer; a gyroscope; a magnetometer; an integrated transceiver for land navigation; and an audio transducer.

At least one of the second set of sensing devices may comprise a machine learning processing device configured for at least one of sensor fusion for sensor data from two or more different types of sensors, object identification, and threat early warning.

Enabling the data sharing of the physiologic monitoring data obtained from the first set of sensing devices and the contextual monitoring data obtained from the second set of sensing devices utilizing the body area network associated with the subject may comprise utilizing at least one of ultrawideband radio communications, Bluetooth radio communications, Bluetooth Low Energy radio communications, long range (LoRa) radio communications, Wifi radio communications, and Near Field Communication (NFC) radio communications.

Pairing the second set of sensing devices with the body area network associated with subject may comprise dynamically pairing respective ones of the second set of sensing devices with the body area network associated with the subject. Dynamically pairing a given one of the second set of sensing devices with the body area network associated with the subject may be performed responsive to identifying that a given one of the one or more microenvironmental monitoring parameters requires sensing data from a sensor type that is available on the given one of the second set of sensing devices but is not available from the first set of sensing devices. The first set of sensing devices may comprise on-body sensing devices and the second set of sensing devices may comprise one or more off-body accessory devices.

In another embodiment, a method performed by at least one processing device implementing a body area network controller for a body area network associated with a subject comprises pairing a first set of sensing devices with the body area network associated with the subject, the first set of sensing devices configured for physiologic monitoring of the subject, and pairing a second set of sensing devices with the body area network associated with the subject, the second set of sensing devices configured for contextual monitoring of an environment of the subject. The method also comprises enabling data sharing of physiologic monitoring data obtained from the first set of sensing devices and contextual monitoring data obtained from the second set of sensing devices utilizing the body area network associated with the subject. The method further comprises determining, based at least in part on the physiologic monitoring data obtained from the first set of sensing devices and the contextual monitoring data obtained from the second set of sensing devices, one or more microenvironmental monitoring parameters associated with the subject.

Pairing the second set of sensing devices with the body area network associated with subject may comprise dynamically pairing respective ones of the second set of sensing devices with the body area network associated with the subject. Dynamically pairing a given one of the second set of sensing devices with the body area network associated with the subject may be performed responsive to identifying that a given one of the one or more microenvironmental monitoring parameters requires sensing data from a sensor type that is available on the given one of the second set of sensing devices but is not available from the first set of sensing devices.

In another embodiment, a computer program product comprises a non-transitory processor-readable storage medium having stored therein executable program code which, when executed, causes at least one processing device implementing a body area network controller for a body area network associated with a subject to perform steps of pairing a first set of sensing devices with the body area network associated with the subject, the first set of sensing devices configured for physiologic monitoring of the subject, pairing a second set of sensing devices with the body area network associated with the subject, the second set of sensing devices configured for contextual monitoring of an environment of the subject, enabling data sharing of physiologic monitoring data obtained from the first set of sensing devices and contextual monitoring data obtained from the second set of sensing devices utilizing the body area network associated with the subject, and determining, based at least in part on the physiologic monitoring data obtained from the first set of sensing devices and the contextual monitoring data obtained from the second set of sensing devices, one or more microenvironmental monitoring parameters associated with the subject.

Pairing the second set of sensing devices with the body area network associated with subject may comprise dynamically pairing respective ones of the second set of sensing devices with the body area network associated with the subject. Dynamically pairing a given one of the second set of sensing devices with the body area network associated with the subject may be performed responsive to identifying that a given one of the one or more microenvironmental monitoring parameters requires sensing data from a sensor type that is available on the given one of the second set of sensing devices but is not available from the first set of sensing devices.

### BRIEF DESCRIPTION OF THE DRAWINGS

Several aspects of the disclosure can be better understood with reference to the following drawings. In the drawings, like reference numerals designate corresponding parts throughout the several views.

FIG. 1 illustrates aspects of a modular physiologic monitoring system, according to an embodiment of the invention.

FIGS. 2A-2D illustrate a modular physiologic monitoring system, according to an embodiment of the invention.

FIGS. 3A-3E illustrate a wearable sensor system configured for monitoring and modeling health data, according to an embodiment of the invention.

FIG. 4 illustrates a system with a body area network controller configured to pair and manage data sharing amongst different types of devices that are part of a body area network associated with a user and one or more external devices, according to an embodiment of the invention.

FIG. 5 illustrates a process flow for microenvironmental monitoring utilizing different types of sensing devices in a body area network associated with a subject, according to an embodiment of the invention.

### DETAILED DESCRIPTION

Particular embodiments of the present disclosure are described herein below with reference to the accompanying drawings; however, the disclosed embodiments are merely examples of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

The accompanying drawings illustrate various embodiments of systems, methods, and embodiments of various other aspects of the disclosure. One of ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. It is also noted that components and elements in the figures are not necessarily drawn to scale, emphasis instead being placed upon illustrating principles.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

One illustrative, non-limiting objective of this disclosure is to provide systems, devices, methods, and kits for monitoring physiologic and/or physical signals from a subject. Another illustrative, non-limiting objective of this disclosure is to provide systems, devices, and methods for managing networks, including body area networks including different types of devices configured for monitoring physiologic and/or physical signals from a subject as well as contextual and environmental information regarding an environment that the subject is in. Another illustrative, non-limiting objective is to provide a flexible architecture enabling sharing of contextual and environmental information amount different types of devices that are part of a body area network associated with a subject. Yet another illustrative, non-limiting objective is to provide systems, devices, and methods for physiologic monitoring of subjects, including physiologic monitoring utilizing contextual and environmental information shared amongst different types of sensing devices that are part of a body area network associated with a subject. Yet another illustrative, non-limiting objective is to provide systems for facilitating interaction between a user and a subject with regard to physiologic and/or environmental monitoring of the subject.

The above illustrative, non-limiting objectives are wholly or partially met by devices, systems, and methods according to the appended claims in accordance with the present disclosure. Features and aspects are set forth in the appended claims, in the following description, and in the annexed drawings in accordance with the present disclosure.

A modular physiologic monitoring system in accordance with the present disclosure is configured to monitor one or more physiologic and/or physical signals, also referred to herein as physiologic parameters, of a subject (e.g., a human subject, a patient, an athlete, a trainer, an animal such as equine, canine, porcine, bovine, etc.). The modular physiologic monitoring system may include one or more patches, each patch adapted for attachment to the body of the subject (e.g., attachable to the skin thereof, reversibly attachable, adhesively attachable, with a disposable interface and a reusable module, etc.). In aspects, the physiologic monitoring system may also include one or more modules, configured and dimensioned to mate with corresponding ones of the one or more patches, and to interface with the subject therethrough. One or more of the modules may be configured to convey and/or store one or more physiologic and/or physical signals, signals derived therefrom, and/or metrics derived therefrom obtained via the interface with the subject.

Each module may include a power source (e.g., a battery, a rechargeable battery, an energy harvesting transducer, microcircuit, an energy reservoir, a thermal gradient harvesting transducer, a kinetic energy harvesting transducer, a radio frequency energy harvesting transducer, a fuel cell, a biofuel cell, etc.), signal conditioning circuitry, communication circuitry, one or more sensors, or the like, configured to generate one or more signals (e.g., physiologic and/or physical signals), stimulus, etc.

One or more of the patches may include one or more interconnects, configured and dimensioned so as to couple with one or more of the modules, said modules including a complementary interconnect configured and dimensioned to couple with the corresponding patch. The patch may include a bioadhesive interface for attachment to the subject, the module retainable against the subject via interconnection with the patch.

In aspects, the patch may be configured so as to be single use (e.g., disposable). The patch may include a thin, breathable, stretchable laminate. In aspects, the laminate may include a substrate, a bioadhesive, one or more sensing or stimulating elements in accordance with the present disclosure, and one or more interconnects for coupling one or more of the sensing elements with a corresponding module.

In aspects, to retain a high degree of comfort and long term wearability of the patch on a subject, to limit interference with normal body function, to limit interference with joint movement, or the like, the patch may be sufficiently thin and frail, such that it may not substantially retain a predetermined shape while free standing. Such a definition is described in further detail below. The patch may be provided with a temporary stiffening film to retain the shape thereof prior to placement of the patch onto the body of a subject. Once adhered to the subject, the temporary stiffening film may be removed from the patch. While the patch is adhered to the subject, the shape and functionality of the patch may be substantially retained. Upon removal of the patch from the subject, the now freestanding patch is sufficiently frail such that the patch can no longer substantially retain the predetermined shape (e.g., sufficiently frail such that the patch will not survive in a free standing state). In aspects, stretch applied to the patch while removing the patch from the subject may result in snap back once the patch is in a freestanding state that renders such a patch to crumple into a ball and no longer function. Removal of the patch from the skin of the subject may result in a permanent loss in shape of the patch without tearing of the patch. In aspects, the interconnect may be sufficiently frail such that removal of the patch from the skin of the subject may result in a permanent loss of shape of the interconnect.

In aspects, the patch may include a film (e.g., a substrate), with sufficiently high tear strength, such that, as the patch is peeled from the skin of a subject, the patch does not tear. In aspects, the ratio between the tear strength of the patch and the peel adhesion strength of the patch to skin (e.g., tear strength: peel adhesion strength), is greater than 8:1, greater than 4:1, greater than 2:1, or the like. Such a configuration may be advantageous so as to ensure the patch may be easily and reliably removed from the subject after use without tearing.

In aspects, the patch may include a bioadhesive with peel tack to mammalian skin of greater than 0.02 Newtons per millimeter (N/mm), greater than 0.1 N/mm, greater than 0.25 N/mm, greater than 0.50 N/mm, greater than 0.75 N/mm, greater than 2 N/mm, or the like. Such peel tack may be approximately determined using an American Society for Testing and Materials (ASTM) standard test, ASTM D3330: Standard test method for peel adhesion of pressure-sensitive tape.

In aspects, the patch may exhibit a tear strength of greater than 0.5 N/mm, greater than 1 N/mm, greater than 2 N/mm, greater than 8 N/mm, or the like. Such tear strength may be approximately determined using an ASTM standard test, ASTM D624: Standard test method for tear strength of conventional vulcanized rubber and thermoplastic elastomers. In aspects, a patch in accordance with the present disclosure may have a ratio between the tear strength of the patch and the peel tack of the adhesive to mammalian skin is greater than 8:1, greater than 4:1, greater than 2:1, or the like.

In aspects, the patch may be provided with a characteristic thickness of less than 50 micrometer (μm), less than 25 μm, less than 12 μm, less than 8 μm, less than 4 μm, or the like. Yet, in aspects, a balance between the thickness, stiffness, and tear strength may be obtained so as to maintain sufficiently high comfort levels for a subject, minimizing skin stresses during use (e.g., minimizing skin stretch related discomfort and extraneous signals as the body moves locally around the patch during use), minimizing impact on skin health, minimizing risk of rucking during use, and minimizing risk of maceration to the skin of a subject, while limiting risk of tearing of the patch during removal from a subject, etc.

In aspects, the properties of the patch may be further altered so as to balance the hydration levels of one or more hydrophilic or amphiphilic components of the patch while attached to a subject. Such adjustment may be advantageous to prevent over hydration or drying of an ionically conducting component of the patch, to manage heat transfer coefficients within one or more elements of the patch, to manage salt absorption into a reservoir in accordance with the present disclosure, and/or migration during exercise, to prevent pooling of exudates, sweat, or the like into a fluid measuring sensor incorporated into the patch or associated module, etc. In aspects, the patch or a rate determining component thereof may be configured with a moisture vapor transmission rate of between 200 grams per meter squared per 24 hours (g/m²/24 hrs) and 20,000 g/m²/24 hrs, between 500 g/m²/24 hrs and 12,000 g/m²/24 hrs, between 2,000 g/m²/24 hrs and 8,000 g/m²/24 hrs, or the like.

Such a configuration may be advantageous for providing a comfortable wearable physiologic monitor for a subject, while reducing material waste and/or cost of goods, preventing contamination or disease spread through uncontrolled re-use, and the like.

In aspects, one or more patches and/or modules may be configured for electrically conducting interconnection, inductively coupled interconnection, capacitively coupled interconnection, with each other. In the case of an electrically conducting interconnect, each patch and module interconnect may include complementary electrically conducting connectors, configured and dimensioned so as to mate together upon attachment. In the case of an inductively or capacitively coupled interconnect, the patch and module may include complementary coils or electrodes configured and dimensioned so as to mate together upon attachment.

Each patch or patch-module pair may be configured as a sensing device to monitor one or more local physiologic and/or physical parameters of the attached subject (e.g., local to the site of attachment, etc.), local environment, combinations thereof, or the like, and to relay such information in the form of signals to a host device (e.g., via a wireless connection, via a body area network connection, or the like), one or more patches or modules on the subject, or the like. Each patch and/or patch-module pair may also or alternatively be configured as a stimulating device to apply a stimulus to the subject in response to signaling from the host device, the signaling being based on analysis of the physiologic and/or physical parameters of the subject measured by the sensing device(s).

The patch or patch-module pairs are examples of what are more generally referred to herein as “primary” sensing devices, which are advantageously designed as on-body sensing devices with a small form factor as part of the modular physiologic monitoring system. While such primary sensing devices may be used to obtain some desired information (e.g., local physiologic and/or physical parameters of the attached subject, local environment, combinations thereof, etc.), in some cases it is beneficial to obtain contextual information from other types of sensors which are difficult to integrate into such primary sensing devices designed as on-body sensing devices with small form factors. Such other types of sensors may be integrated into “secondary” or accessory sensing devices that do not have the limitations of the “primary” sensing devices. For example, while the primary sensing devices may be designed as on-body sensing devices with a small form factor for comfortable long-term wear by the subject, the secondary or accessory sensing devices may have larger form factors to accommodate different types of sensors than the primary sensing devices. It should be noted that the secondary or accessory sensing devices may be incorporated into equipment or gear that is carried by a subject, into one or more wearable computing devices, etc. In some cases, an accessory sensing device is directly attached to the body of the subject.

The on-body physiologic monitoring or other primary sensing devices can benefit from additional contextual and environmental information about the conditions surrounding a subject under study, where the additional contextual and environmental information may be obtained from one or more accessory sensing devices. For example, the primary sensing devices may be used to acquire one or more physiologic metrics such as heart rate, core temperature, etc. Such physiologic metric data may be augmented by contextual or environmental data obtained using additional external sensing capabilities of accessory sensing devices, where the accessory sensing devices may target exposure to infectious agents, insolation, etc. This contextualization capability may, under some circumstances, need to be flexible, requiring different sensing modalities at different times with different subjects under study. In addition, some sensors may not be easily integrated into a single primary (e.g., on-body) sensing device with a small form factor, and thus may need to be externalized into one or more accessory sensing devices that may be placed at different locations relative to the primary sensing devices on the same individual. These various primary and accessory sensing devices may require a dedicated BAN to manage their functions, to enable efficient data sharing among them, and to facilitate contextual analysis of the different data obtained therefrom.

In aspects, the host device may be configured to coordinate information exchange to/from each module and/or patch or other on-body primary sensing device as well as accessory sensing devices that are part of a BAN associated with a subject, and to generate one or more physiologic signals, physical signals, environmental signals, kinetic signals, diagnostic signals, alerts, reports, recommendation signals, commands, combinations thereof, or the like for the subject, a user, a network, an electronic health record (EHR), a database (e.g., as part of a data management center, an EHR, a social network, etc.), a processor, combinations thereof, or the like. In aspects, the host device may include features for recharging and/or performing diagnostic tests on one or more of the modules. In aspects, a host device in accordance with the present disclosure may be integrated into a bedside alarm clock, housed in an accessory, within a purse, a backpack, a wallet, or may be included in a mobile computing device, a smartphone, a tablet computer, a pager, a laptop, a local router, a data recorder, a network hub, a server, a secondary mobile computing device, a repeater, a combination thereof, or the like.

In aspects, a system in accordance with the present disclosure may include a plurality of substantially similar modules (e.g., generally interchangeable modules, but with unique identifiers), for coupling with a plurality of patches, each patch, optionally different from the other patches in the system (e.g., potentially including alternative sensors, sensor types, sensor configurations, electrodes, electrode configurations, etc.). Each patch may include an interconnect suitable for attachment to an associated module. Upon attachment of a module to a corresponding patch, the module may validate the type and operation of the patch to which it has been mated. In aspects, the module may then initiate monitoring operations on the subject via the attached patch, communicate with one or more other patches on the subject, a hub, etc. The data collection from each module may be coordinated through one or more modules and/or with a host device in accordance with the present disclosure. The modules may report a timestamp along with the data in order to synchronize data collection across multiple patch-module pairs on the subject, between subjects, etc. Thus, if a module is to be replaced, a hot swappable replacement (e.g., replacement during a monitoring procedure) can be carried out easily by the subject, a caregiver, practitioner, etc., during the monitoring process. Such a configuration may be advantageous for performing redundant, continuous monitoring of a subject, and/or to obtain spatially relevant information from a plurality of locations on the subject during use.

One or more devices in the network may include a time synchronization service, the time synchronization service configurable so as to periodically align the local time sources of each device to those of each of the other devices in the network. In aspects, the time synchronization may be performed every second, every ten seconds, every thirty seconds, every minute or the like. In aspects, one or more local devices may be coupled to an external time source such as an internet accessible time protocol, or a geolocation-based time source. Such information may be brought into the network so as to help align a global time reference for devices in the network. Such information may propagate through the network devices using the time synchronization service.

In a time aligned configuration, one or more metrics measured from a subject in connection with one or more devices in the network may be time aligned with one or more metrics from a different subject in the network. As such, events that can simultaneously affect multiple subjects can be registered and higher level event classification algorithms are configured so as to generate an appropriate alert based on the metrics measured.

In aspects, an event may include a loud audible event, or a physiological response to an event, the event classification algorithm is configured so as to increase the priority of an alert if the number of subjects affected by the event increases beyond a set number.

In aspects the modules and/or patches may include corresponding interconnects for coupling with each other during use. The interconnects may include one or more connectors, configured such that the modules and patches may only couple in a single unique orientation with respect to each other. In aspects, the modules may be color coded by function. A temporary stiffening element attached to a patch may include instructions, corresponding color coding, etc., so as to assist a user or subject with simplifying the process of monitoring.

In addition to physiologic monitoring, one or more patches and/or modules may be used to provide a stimulus to the subject, as will be described in further detail below.

According to aspects there is provided use of a modular physiologic monitoring system in accordance with the present disclosure to monitor a subject, to monitor an electrocardiogram (EKG) of a subject, to perform one or more tasks in accordance with the present disclosure, etc.

According to aspects there is provided an interface (e.g., a patch in accordance with the present disclosure) for monitoring a physiologic, physical, and/or electrophysiological signal from a subject. The interface or patch may include a substrate, an adhesive coupled to the substrate formulated for attachment to the skin of a subject, and one or more sensors and/or electrodes each in accordance with the present disclosure coupled to the substrate, arranged, configured, and dimensioned to interface with the subject. The substrate may be formed from an elastic or polymeric material, such that the patch is configured to maintain operation when stretched to more than 25%, more than 50%, or more than 80%.

According to aspects there is provided an isolating patch for providing a barrier between a handheld monitoring device with a plurality of contact pads and a subject, including a flexible substrate with two surfaces, a patient facing surface and an opposing surface, and an electrically and/or ionically conducting adhesive coupled to at least a portion of the patient facing surface configured so as to electrically and mechanically couple with the subject when placed thereupon, wherein the conducting adhesive is exposed within one or more regions of the opposing surface of the substrate, the regions patterned so as to substantially match the dimensions and layout of the contact pads. In aspects, the conducting adhesive may include an anisotropically conducting adhesive, with the direction of conduction oriented substantially normal to the surfaces of the substrate.

In aspects, the adhesive may be patterned onto the substrate so as to form one or more exposed regions of the substrate, one or more of the sensors and/or electrodes arranged within the exposed regions. One or more of the electrodes may include an inherently or ionically conducting gel adhesive.

In aspects, one or more of the electrodes may include an electrode feature arranged so as to improve the electrical connection between the electrode and the skin upon placement on a subject. In aspects, the improved electrical connection may be achieved after pressure is applied to the electrode (e.g., after the patch is secured to the subject and then a pressure is applied to the electrode). The electrode feature may include one or more microfibers, barbs, microneedles, or spikes to penetrate into a stratum corneum of the skin. The electrode feature may be configured to penetrate less than 2 mm into the skin, less than 1 mm, less than 0.5 mm, less than 0.2 mm, or the like during engagement therewith. In aspects, a gel adhesive in accordance with the present disclosure located adjacent to the electrode features (e.g., between the features and the skin) may be configured to maintain the improved electrical connection to the skin for more than 1 hour, more than 1 day, or more than 3 days after the electrode contacts the skin or pressure is applied to the electrode.

In aspects, a patch interface in accordance with the present disclosure may include one or more stretchable electrically conducting traces attached to the substrate, arranged so as to couple one or more of the sensors and/or electrodes with one or more of the interconnects.

In aspects, the interconnect may include a plurality of connectors, the connectors physically connected to each other through the substrate. The patch may include an isolating region arranged so as to isolate one or more of the connectors from the skin while the patch is engaged therewith.

According to aspects there is provided a device (e.g., a module in accordance with the present disclosure) for monitoring a physiologic, physical, and/or electrophysiological signals from a subject. The module may include a housing, a printed circuit board (PCB) including one or more microcircuits, and an interconnect configured for placement of the device onto a subject interface (e.g., a patch in accordance with the present disclosure). The PCB may constitute at least a portion of the housing in some embodiments. The module may include a three-dimensional antenna coupled to the microcircuits (e.g., coupled with a transceiver, transmitter, radio, etc., included within the microcircuits). In aspects, the antenna may be printed onto or embedded into the housing. In aspects, the antenna may be printed on an interior wall of or embedded into the housing, the circuit board providing a ground plane for the antenna. In aspects, the housing may be shaped like a dome and the antenna may be patterned into a spiraling helix centered within the dome.

In aspects, a module in accordance with the present disclosure may include a sensor coupled with one or more of the microcircuits, the sensor configured to interface with the subject upon attachment of the module to the patch. The module may include a sensor and/or microelectronics configured to interface with a sensor included on a corresponding patch. In aspects, one or more of the sensors may include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, a combination thereof, or the like.

In aspects, the module may be hermetically sealed. The module and/or patch may include a gasket coupled to the circuit board or the substrate, the gasket formed so as to isolate the region formed by the module interconnect and the patch from a surrounding environment, when the module is coupled with the patch.

In aspects, the module interconnect may include an electrically conducting magnetic element, and the patch may include one or more ferromagnetic regions coupled to the substrate, the magnetic elements arranged so as to physically and/or electrically couple the module to the patch when the magnetic elements are aligned with the ferromagnetic regions. In aspects, the ferromagnetic regions may be formed from stretchable pseudo elastic material and/or may be printed onto the substrate. In aspects, the module and/or the patch may include one or more fiducial markings to visually assist with the alignment of the module to the patch during coupling thereof.

According to aspects there is provided a kit for monitoring one or more physiologic, physical, and/or electrophysiological signals from a subject, including one or more patches in accordance with the present disclosure, one or more modules in accordance with the present disclosure, a recharging bay in accordance with the present disclosure, and one or more accessories in accordance with the present disclosure. One or more of the accessories may include an adhesive removing agent configured to facilitate substantially pain free removal of one or more of the patches from a subject. One or more other ones of the accessories may include accessory sensing device configured to complement (e.g., provide contextual or environmental information) that augments physiologic data obtained from patches and/or patch-module pairs providing primary sensing devices.

According to aspects there is provided a service system for managing the collection of physiologic data from a customer, including a customer data management service, configured to generate and/or store the customer profile referencing customer preferences, data sets, and/or monitoring sessions, an automated product delivery service configured to provide the customer with one or more monitoring products or supplies in accordance with the present disclosure, and a datacenter configured to store, analyze, and/or manage the data obtained from the customer during one or more monitoring sessions.

In aspects, the service system may include a report generating service configured to generate one or more monitoring reports based upon the data obtained during one or more monitoring sessions, a report generating service coupled to the datacenter configured to generate one or more monitoring reports based upon the data obtained during one or more monitoring sessions, and/or a recurrent billing system configured to bill the customer based upon the number or patches consumed, the data stored, and/or the reports generated throughout the course of one or more monitoring sessions.

According to aspects there is provided a method for monitoring one or more physiologic and/or electrophysiological signals from a subject, including attaching one or more soft, breathable and hypoallergenic devices to one or more sites on the subject, obtaining one or more local physiologic and/or electrophysiological signals from each of the devices, obtaining contextual or environmental information from secondary or accessory sensing devices, and analyzing the signals obtained from the primary and secondary sensing devices to generate a metric, diagnostic, report, and/or additional signals therefrom.

In aspects, the method may include hot swapping one or more of the devices without interrupting the step of obtaining, and/or calibrating one or more of the devices while on the subject. In aspects, the step of calibrating may be performed with an additional medical device (e.g., a blood pressure cuff, a thermometer, a pulse oximeter, a cardiopulmonary assessment system, a clinical grade EKG diagnostic system, etc.).

In aspects, the method may include determining the position and/or orientation of one or more of the devices on the subject, and/or determining the position and/or orientation from a photograph, a video, or a surveillance video.

In aspects, one or more steps of a method in accordance with the present disclosure may be performed at least in part by one or more devices, patches, modules, and/or systems each in accordance with the present disclosure.

According to aspects there is provided a system for measuring blood pressure of a subject in an ambulatory setting including an EKG device in accordance with the present disclosure (e.g., a patch/module pair in accordance with the present disclosure configured to measure local electrophysiological signals in adjacent tissues), configured for placement onto a torso of the subject, the EKG device configured to measure an electrocardiogramal from the torso of the subject so as to produce an EKG signal, one or more pulse devices (e.g., patch/module pairs in accordance with the present disclosure configured to measure local blood flow in adjacent tissues) each in accordance with the present disclosure, configured for placement onto one or more sites on one or more extremities of the subject, each of the pulse devices configured to measure a local pulse at the placement site so as to produce one or more pulse signals; and a processor included in or coupled to one or more of the EKG device and the pulse devices, the processor configured to receive the EKG signal, the pulse signals, and/or signals generated therefrom, the processor including an algorithm, the algorithm configured to analyze one or more temporal metrics from the signals in combination with one or more calibration parameters, to determine the blood pressure of the subject.

In aspects, the system for monitoring blood pressure of a subject may include a blood pressure cuff configured to produce a calibration signal, the processor configured to generate one or more of the calibration parameters, from the calibration signal in combination with the EKG signal, and pulse signals.

In aspects, one or more of the devices may include an orientation sensor, the orientation sensor configured to obtain an orientation signal, the processor configured to receive the orientation signal or a signal generated therefrom, and to incorporate the orientation signal into the analysis. Some non-limiting examples of orientation sensors include one or more of an altimeter, a barometer, a tilt sensor, a gyroscope, combinations thereof, or the like.

A system for measuring the effect of an impact on physiologic state of a subject including an electroencephalogram (EEG) device (e.g., a patch/module pair in accordance with the present disclosure configured to measure local electrophysiological signals associated with brain activity in adjacent tissues) in accordance with the present disclosure, configured for placement behind an ear, on the forehead, near a temple, onto the neck of the subject, or the like, the EEG device configured to measure an electroencephalographic signal from the head of the subject so as to produce an EEG signal, and configured to measure one or more kinetic and/or kinematic signals from the head of the subject so as to produce an impact signal, and a processor included in or coupled to the EEG device, the processor configured to receive the EEG signal, the impact signals, and/or signals generated therefrom, the processor including an algorithm, the algorithm configured to analyze the impact signals to determine if the subject has suffered an impact, to separate the signals into pre impact and post impact portions and to compare the pre and post impact portions of the EEG signal, to determine the effect of the impact on the subject.

In aspects, the EEG device may include additional sensors such as a temperature sensor configured to generate a temperature signal from the subject or a signal generated therefrom, the processor configured to receive the temperature signal and to assess a thermal state of the subject therefrom. In aspects, the EEG device may include a hydration sensor configured to generate a fluid level signal from the subject, the processor configured to receive the fluid level signal or a signal generated therefrom, and to assess the hydration state of the subject therefrom.

In aspects, the EEG device and/or the processor may include or be coupled to a memory element, the memory element including sufficiently large space to store the signals for a period of 3 minutes, 10 minutes, 30 minutes, or 1 hour.

In aspects, the system for measuring the effect of an impact on physiologic state of a subject may include an EKG device (e.g., a patch/module pair in accordance with the present disclosure configured to measure local electrophysiological signals in adjacent tissues) in accordance with the present disclosure, the EKG device configured for placement onto the torso or neck of the subject, the EKG device configured to measure an electrophysiological signal pertaining to cardiac function of the subject so as to produce an EKG signal, the processor configured to receive the EKG signal or a signal generated therefrom, the algorithm configured so as to incorporate the EKG signal into the assessment. In aspects, the processor may be configured to extract a heart rate variability (HRV) signal from the EKG signal, to compare a pre impact and post impact portion of the HRV signal to determine at least a portion of the effect of the impact, etc.

According to aspects there is provided a system for assessing a sleep state of a subject including an electromyography (EMG)/electrooculography (EOG) device (e.g., a patch/module pair in accordance with the present disclosure configured to measure local electromyographic and/or electrooculographic signals from adjacent tissues), in accordance with the present disclosure, configured for placement behind an ear, on a forehead, substantially around an eye, near a temple, or onto a neck of the subject, the EMG/EOG device configured to measure one or more electromyographic and/or electrooculographic signals from the head or neck of the subject so as to produce an EMG/EOG signal, and a processor included in or coupled to the EMG/EOG device, the processor configured to receive the EMG/EOG signal, and/or signals generated therefrom, the processor including an algorithm, the algorithm configured to analyze EMG/EOG signal, to determine the sleep state of the subject.

In aspects, the EMG/EOG device may include a microphone, the microphone configured to obtain an acoustic signal from the subject, the processor configured to receive the acoustic signal or a signal generated therefrom, the algorithm configured so as to incorporate the acoustic signal into the assessment.

In aspects, the system may include a sensor for evaluating oxygen saturation (SpO2) at one or more sites on the subject to obtain an oxygen saturation signal from the subject, the processor configured to receive the oxygen saturation signal or a signal generated therefrom, the algorithm configured so as to incorporate the oxygen saturation signal into the assessment.

In aspects, the processor may include a signal analysis function, the signal analysis function configured to analyze the EMG/EOG signals, the acoustic signal, and/or the oxygen saturation signal to determine the sleep state of the subject, to identify snoring, to identify a sleep apnea event, to identify a bruxism event, to identify a rapid eye movement (REM) sleep state, to identify a sleep walking state, a sleep talking state, a nightmare, or to identify a waking event. In aspects, the system may include a feedback mechanism, configured to interact with the subject, a user, a doctor, a nurse, a partner, a combination thereof, or the like. The processor may be configured to provide a feedback signal to the feedback mechanism based upon the analysis of the sleep state of the subject. The feedback mechanism may include a transducer, a loudspeaker, tactile actuator, a visual feedback means, a light source, a buzzer, a combination thereof, or the like to interact with the subject, the user, the doctor, the nurse, the partner, or the like.

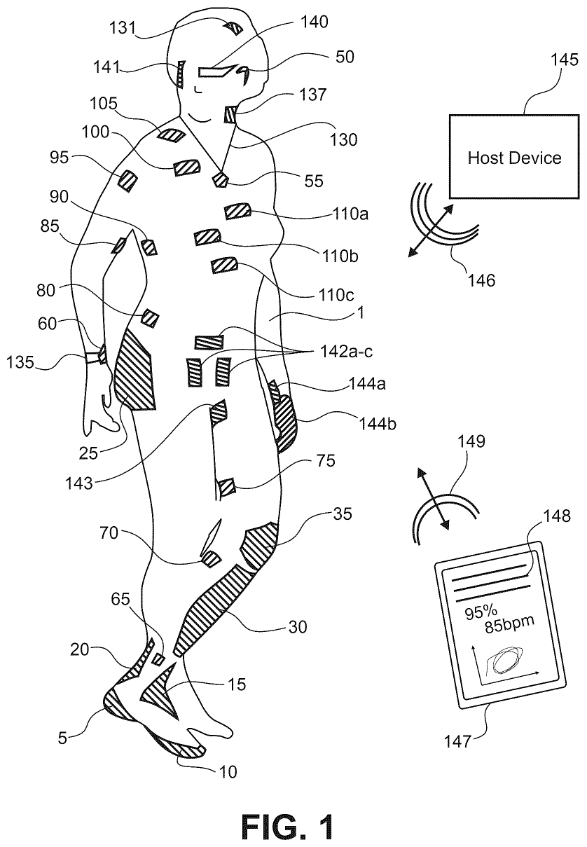

A modular physiologic monitoring system, in some embodiments, includes one or more sensing devices, which may be placed or attached to one or more sites on the subject. Alternatively, or additionally, one or more sensing devices may be placed “off”′ the subject, such as one or more sensors (e.g., cameras, acoustic sensors, etc.) that are not physically attached to the subject. The sensing devices are utilized to establish whether or not an event is occurring and to determine one or more characteristics of the event by monitoring and measuring physiologic parameters of the subject. The determination of whether an event has occurred or is occurring may be made by a device that is at least partially external and physically distinct from the one or more sensing devices, such as a host device in wired or wireless communication with the sensing devices as described below with respect to FIG. 1. The modular physiologic monitoring system may include one or more stimulating devices, which again may be any combination of devices that are attached to the subject or placed “off” the subject, to apply a stimulus to the subject in response to a detected event. Various types of stimulus may be applied, including but not limited to stimulating via thermal input, vibration input, mechanical input, a compression or the like with an electrical input, etc.

The sensing devices of a modular physiologic monitoring system, such as patch-module pairs described below with respect to FIG. 1, may be used to monitor one or more physiologic functions or parameters of a subject, as will be described in further detail below. The sensing devices of the modular physiologic monitoring system, or a host device configured to receive data or measurements from the sensing devices, may be utilized to monitor for one or more events (e.g., through analysis of signals measured by the sensing devices, from metrics derived from the signals, etc.). The stimulating devices of the modular physiologic monitoring system may be configured to deliver one or more stimuli (e.g., electrical, vibrational, acoustic, visual, etc.) to the subject. The stimulating devices may receive a signal from one or more of the sensing devices or a host device, and provide the stimulation in response to the received signal.

FIG. 1 shows aspects of a modular physiologic monitoring system in accordance with the present disclosure. In FIG. 1, a subject 1 is shown with a number of patches and/or patch-module pairs each in accordance with the present disclosure attached thereto at sites described below, a host device 145 in accordance with the present disclosure, a feedback/user device 147 in accordance with the present disclosure displaying some data 148 based upon signals obtained from the subject 1, and one or more feedback devices 135, 140, in accordance with the present disclosure configured to convey to the subject 1 one or more aspects of the signals or information gleaned therefrom. In some embodiments, the feedback devices 135, 140 may also or alternatively function as stimulating devices. The host device 145, the user device 147, the patches and/or patch-module pairs, and/or the feedback devices 135, 140 may be configured for wireless communication 146, 149 during a monitoring session.

In aspects, a patch-module pair may be adapted for placement almost anywhere on the body of a subject 1. As shown in FIG. 1, some sites may include attachment to the cranium or forehead 131, the temple, the ear or behind the ear 50, the neck, the front, side, or back of the neck 137, a shoulder 105, a chest region with minimal muscle mass 100, integrated into a piece of ornamental jewelry 55 (may be a host, a hub, a feedback device, etc.), arrangement on the torso 110a-c, arrangement on the abdomen 80 for monitoring movement or breathing, below the rib cage 90 for monitoring respiration (generally on the right side of the body to substantially reduce EKG influences on the measurements), on a muscle such as a bicep 85, on a wrist 135 or in combination with a wearable computing device 60 on the wrist (e.g., a smart watch, a fitness band, etc.), on a buttocks 25, on a thigh 75, on a calf muscle 70, on a knee 35 particularly for proprioception based studies and impact studies, on a shin 30 primarily for impact studies, on an ankle 65, over an Achilles tendon 20, on the front or top of the foot 15, on a heel 5, or around the bottom of a foot or toes 10. Other sites for placement of such devices are envisioned. Selection of the monitoring and/or stimulating sites is generally determined based upon the intended application of the patch-module pairs described herein.

Additional placement sites on the abdomen, perineal region 142a-c, genitals, urogenital triangle, anal triangle, sacral region, inner thigh 143, or the like may be advantageous in the assessment of autonomic neural function of a subject. Such placements regions may be advantageous for assessment of parasympathetic nervous system (PNS) activity, somatosensory function, assessment of sympathetic nervous system (SNS) functionality, etc.

Placement sites on the wrist 144a, hand 144b or the like may advantageous for interacting with a subject, such as via performing a stress test, performing a thermal stress test, performing a tactile stress test, monitoring outflow, afferent traffic, efferent traffic, etc.

Placement sites on the nipples, areola, lips, labia, clitoris, penis, the anal sphincter, levator ani muscle, over the ischiocavernous muscle, deep transverse perineal muscle, labium minus, labium majus, one or more nerves near the surface thereof, posterior scrotal nerves, perineal membrane, perineal nerves, superficial transverse perineal nerves, dorsal nerves, inferior rectal nerves, etc., may be advantageous for assessment of autonomic neural ablation procedures, autonomic neural modulation procedures, assessment of the PNS of a subject, assessment of sexual dysfunction of a subject, etc.

Placement sites on the face 141, over ocular muscles, near the eye, over a facial muscle (e.g., a nasalis, temporalis, zygomaticus minor/major, orbicularis oculi, occipitofrontalis), near a nasal canal, over a facial bone (e.g., frontal process, zygomatic bone/surface, zygomaticofacial foreman, malar bone, nasal bone, frontal bone, maxilla, temporal bone, occipital bone, etc.), may be advantageous to assess ocular function, salivary function, sinus function, interaction with the lips, interaction with one or more nerves of the PNS (e.g., interacting with the vagus nerve within, on, and/or near the ear of the subject), etc.

In aspects, a system in accordance with the present disclosure may be configured to monitor one or more physiologic parameters of the subject 1 before, during, and/or after one or more of, a stress test, consumption of a medication, exercise, a rehabilitation session, a massage, driving, a movie, an amusement park ride, sleep, intercourse, a surgical, interventional, or non-invasive procedure, a neural remodeling procedure, a denervation procedure, a sympathectomy, a neural ablation, a peripheral nerve ablation, a radio-surgical procedure, an interventional procedure, a cardiac repair, administration of an analgesic, a combination thereof, or the like. In aspects, a system in accordance with the present disclosure may be configured to monitor one or more aspects of an autonomic neural response to a procedure, confirm completion of the procedure, select candidates for a procedure, follow up on a subject after having received a procedure, assess the durability of a procedure, or the like (e.g., such as wherein the procedure is a renal denervation procedure, a carotid body denervation procedure, a hepatic artery denervation procedure, a LUTs treatment, a bladder denervation procedure, a urethral treatment, a prostate ablation, a prostate nerve denervation procedure, a cancer treatment, a pain block, a neural block, a bronchial denervation procedure, a carotid sinus neuromodulation procedure, implantation of a neuromodulation device, tuning of a neuromodulation device, etc.).

Additional details regarding modular physiologic monitoring systems, kits and methods are further described in PCT application serial no. PCT/US2014/041339, published as WO 2014/197822 and titled “Modular Physiologic Monitoring Systems, Kits, and Methods,” PCT application serial no. PCT/US2015/043123, published as WO 2016/019250 and titled “Modular Physiologic Monitoring Systems, Kits, and Methods,” PCT application serial no. PCT/US2017/030186, published as WO 2017/190049 and titled “Monitoring and Management of Physiologic Parameters of a Subject,” PCT application serial no. PCT/US2018/062539, published as WO 2018/098073 and titled “Continuous Long-Term Monitoring of a Subject,” PCT application serial no. PCT/US2018/043068, published as WO 2019/023055 and titled “Physiologic Monitoring Kits,” PCT application serial no. PCT/2019/033036, published as WO 2019/226506 and titled “Monitoring Physiologic Parameters for Timing Feedback to Enhance Performance of a Subject During an Activity,” PCT application serial no. PCT/US2020/031851, published as WO 2020/227514 and titled “Monitoring and Processing Physiological Signals to Detect and Predict Dysfunction of an Anatomical Feature of an Individual,” PCT application serial no. PCT/US2021033441, published as WO 2021/236948 and titled “Gateway Device Facilitating Collection and Management of Data from a Body Area Network to Study Coordinating System,” PCT application serial no. PCT/US2021/028611, published as WO 2021/216847 and titled “Visualizing Physiologic Data Obtained from Subjects,” PCT application serial no. PCT/US2021/033442, published as WO 2021/236949 and titled “Non-Invasive Detection of Anomalous Physiologic Events Indicative of Hypovolemic Shock of a Subject,” PCT application serial no. PCT/US2021/041414, published as WO 2022/015719 and titled “Wearable Sensor System Configured for Monitoring and Modeling Health Data,” PCT application serial no. PCT/US2021041418, published as WO 2022/015722 and titled “Wearable Sensor System Configured for Facilitating Telemedicine Management,” and PCT application serial no. PCT/US2021/041420, published as WO 2022/015724 and titled “Wearable Sensor System Configured for Alerting First Responders and Local Caregivers,” the disclosures of which are incorporated by reference herein in their entirety.

In some embodiments, modular physiologic monitoring systems may include sensing and stimulating devices that are physically distinct, such as sensing and stimulating devices that are physically attached to a subject at varying locations. For example, the sensing and stimulating devices may include different ones of the patch-module pairs described above with respect to FIG. 1. In other embodiments, one or more devices may provide both monitoring and stimulating functionality. For example, one or more of the patch-module pairs described above with respect to FIG. 1 may be configured to function as both a sensing device and a stimulating device. It is to be appreciated, however, that embodiments are not limited solely for use with the patch-module pairs of FIG. 1 as sensing and stimulating devices. Various other types of sensing and stimulating devices may be utilized, including but not limited to sensors that are “off-body” with respect to subject 1.

The sensing and/or stimulating devices of a modular physiologic monitoring system may be configured for radio frequency (RF) or other wireless and/or wired connection with one another and/or a host device. Such RF or other connection may be used to transmit or receive feedback parameters or other signaling between the sensing and stimulating devices. The feedback, for example, may be provided based on measurements of physiologic parameters that are obtained using the sensing devices to determine when events related to cardiac output are occurring. Various thresholds for stimulation that are applied by the stimulating devices may, in some embodiments, be determined based on such feedback. Thresholds may relate to the amplitude or frequency of electric or other stimulation. Thresholds may also be related to whether to initiate stimulation by the stimulating devices based on the feedback.

During and/or after stimulus is applied with the stimulating devices, the sensing devices may monitor the physiologic response of the subject. If stimulation is successful in achieving a desired response, the stimulation may be discontinued. Otherwise, the type, timing, etc., of stimulation may be adjusted.

In some embodiments, a user of the modular physiologic monitoring system may set preferences for the stimulus type, level, and/or otherwise personalize the sensation during a setup period or at any point during use of the modular physiologic monitoring system. The user of the modular physiologic monitoring system may be the subject being monitored and stimulated by the sensing devices and stimulating devices, or a doctor, nurse, physical therapist, medical assistant, caregiver, etc., of the subject being monitored and stimulated. The user may also have the option to disconnect or shut down the modular physiologic monitoring system at any time, such as via operation of a switch, pressure sensation, voice operated instruction, etc.

Stimulus or feedback which may be provided via one or more stimulating devices in modular physiologic monitoring system may be in various forms, including physical stimulus (e.g., electrical, thermal, vibrational, pressure, stroking, a combination thereof, or the like), optical stimulus, acoustic stimulus, etc.

Physical stimulus may be provided in the form of negative feedback, such as in a brief electric shock or impulse as described above. Data or knowledge from waveforms applied in conducted electrical weapons (CEWs), such as in electroshock devices, may be utilized to avoid painful stimulus. Physical stimulus may also be provided in the form of positive feedback, such as in evoking pleasurable sensations by combining non-painful electrical stimulus with pleasant sounds, music, lighting, smells, etc. Physical stimulus is not limited solely to electrical shock or impulses. In other embodiments, physical stimulus may be provided by adjusting temperature or other stimuli, such as in providing a burst of cool or warm air, a burst of mist, vibration, tension, stretch, pressure, etc.

Feedback provided via physical stimulus as well as other stimulus described herein may be synchronized with, initiated by or otherwise coordinated or controlled in conjunction with one or more monitoring devices (e.g., a host device, one or more sensing devices, etc.). The monitoring devices may be connected to the stimulating devices physically (e.g., via one or more wires or other connectors), wirelessly (e.g., via radio or other wireless communication), etc. Physical stimulus may be applied to various regions of a subject, including but not limited to the wrist, soles of the feet, palms of the hands, nipples, forehead, ear, mastoid region, the skin of the subject, etc.

Optical stimulus may be provided via one or more stimulating devices. The optical stimulus may be positive or negative (e.g., by providing pleasant or unpleasant lighting or other visuals). Acoustic stimulus similarly may be provided via one or more stimulating devices, as positive or negative feedback (e.g., by providing pleasant or unpleasant sounds). Acoustic stimulus may take the form of spoken words, music, etc. Acoustic stimulus, in some embodiments may be provided via smart speakers or other electronic devices such as Amazon Echo®, Google Home®, Apple Home Pod®, etc. The stimulus itself may be provided so as to elicit a particular psychophysical or psychoacoustic effect in the subject, such as directing the subject to stop an action, to restart an action (such as breathing), to adjust an action (such as a timing between a step and a respiratory action, between a muscle contraction and a leg position, etc.).

As described above, the modular physiologic monitoring system may operate in a therapeutic mode, in that stimulation is provided when one or more cardiac parameters of a subject indicate some event (e.g., actual, imminent or predicted failure or worsening). The modular physiologic monitoring system, however, may also operate as or provide a type of cardiac “pacemaker” in other embodiments. In such embodiments, the modular physiologic monitoring system has the potential to reduce the frequency of cardiac events, or to possibly avoid certain cardiac events altogether. A modular physiologic monitoring system may provide functionality for timing and synchronizing periodic compression and relaxation of microvascular blood vessel networks with cardiac output. Such techniques may be utilized to respond to a type of failure event as indicated above. Alternatively or additionally, such techniques may be provided substantially continuously, so as to improve overall cardiac performance (e.g., blood flow) with the same or less cardiac work.

In some embodiments, a modular physiologic monitoring system may be configured to provide multi-modal stimuli to a subject. Multi-modal approaches use one or more forms of stimulation (e.g., thermal and electrical, mechanical and electrical, etc.) in order to mimic another stimulus to trick local nerves into responding in the same manner to the mimicked stimulus. In addition, in some embodiments multi-modal stimulus or input may be used to enhance a particular stimulus. For example, adding a mimicked electrical stimulus may enhance the effect of a thermal stimulus.

Modular physiologic monitoring systems may use pulses across space and time (e.g., frequency, pulse trains, relative amplitudes, etc.) to mimic vibration, comfort or discomfort, mild or greater pain, wet sensation, heat/cold, training neuroplasticity, taste (e.g., using a stimulating device placed in the mouth or on the tongue of a subject to mimic sour, sweet, salt, bitter or umami flavor), tension or stretching, sound or acoustics, sharp or dull pressure, light polarization (e.g., linear versus polar, the “Haidinger Brush”), light color or brightness, etc.

Stimulus amplification may also be provided by one or more modular physiologic monitoring systems using multi-modal input. Stimulus amplification represents a hybrid approach, wherein a first type of stimulus may be applied and a second, different type of stimulus provided to enhance the effect of the first type of stimulus. As an example, a first stimulus may be provided via a heating element, where the heating element is augmented by nearby electrodes or other stimulating devices that amplify and augment the heating stimulus using electrical mimicry in a pacing pattern. Electrical stimulus may also be used as a supplement or to mimic various other types of stimulus, including but not limited to vibration, heat, cold, etc. Different, possibly unique, stimulation patterns may be applied to the subject, with the central nervous system and peripheral nervous system interpreting such different or unique stimulation patterns as different stimulus modalities.

Another example of stimulus augmentation is sensing a “real” stimulus, measuring the stimulus, and constructing a proportional response by mimicry such as using electric pulsation. The real stimulus, such as sensing heat or cold from a Peltier device, may be measured by electrical-thermal conversion. This real stimulus may then be amplified using virtual mimicry, which may provide energy savings and the possibility of modifying virtual stimulus to modify the perception of the real stimulus.

In some embodiments, the stimulating devices in a modular physiologic monitoring system include an electrode array that attaches (e.g., via an adhesive or which is otherwise held in place) to a preferred body part. One or more of the stimulating devices may include a multiplicity of both sensing and stimulation electrodes, including different types of sensing and/or stimulation electrodes. The sensing electrodes on the stimulation devices, in some embodiments, may be distinct from the sensing devices in the modular physiologic monitoring system in that the sensing devices in the modular physiologic monitoring system may be used to measure physiologic parameters of the subject while the sensing electrodes on the stimulation devices in the modular physiologic monitoring system may be utilized to monitor the application of a stimulus to the subject.

A test stimulus may be initiated in a pattern in the electrode array, starting from application via one or a few of the stimulation electrodes and increasing in number over time to cover an entire or larger portion of the electrode array. The test stimulus may be used to determine the subject's response to the applied stimulation. Sensing electrodes on the stimulation devices may be used to monitor the application of the stimulus. The electrode array may also be used to record a desired output (e.g., physiologic parameters related to cardiac output). As such, one or more of the electrodes in the array may be configured so as to measure the local evoked response associated with the stimulus itself. Such an approach may be advantageous to confirm capture of the target nerves during use. By monitoring the neural response to the stimulus, the stimulus parameters including amplitude, duration, pulse number, etc., may be adjusted while ensuring that the target nerves are enlisted by the stimulus in use.

The test stimulus may migrate or be applied in a pattern to different electrodes at different locations in the electrode array. The response to the stimulus may be recorded or otherwise measured, using the sensing devices in the modular physiologic monitoring system and/or one or more of the sensing electrodes of the stimulating devices in the modular physiologic monitoring system. The response to the test stimulus may be recorded or analyzed to determine an optimal sensing or application site for the stimulus to achieve a desired effect or response in the subject. Thus, the test stimulus may be utilized to find an optimal sensing (e.g., dermatome driver) location. This allows for powerful localization for optimal pacing or other application of stimulus, which may be individualized for different subjects.

A stimulating device applied to the subject via an adhesive (e.g., an adhesively applied stimulating device), may be in the form of a disposable or reusable unit, such as a patch and or patch-module or patch/hub pair as described above with respect to FIG. 1. An adhesively applied stimulating device, in some embodiments, includes a disposable interface configured so as to be thin, stretchable, able to conform to the skin of the subject, and sufficiently soft for comfortable wear. The disposable interface may be built from very thin, stretchable and/or breathable materials, such that the subject generally does not feel the device on his or her body.

Actuation means of the adhesively applied stimulating device may be applied over a small region of the applied area of the subject, such that the adhesive interface provides the biasing force necessary to counter the actuation of the actuation means against the skin of the subject.

Adhesively applied stimulating devices may be provided as two components-a disposable body interface and a reusable component. The disposable body interface may be applied so as to conform to the desired anatomy of the subject, and wrap around the body such that the reusable component may interface with the disposable component in a region that is open and free from a natural interface between the subject and another surface.

An adhesively applied stimulating device may also be a single component, rather than a two component or other multi-component arrangement. Such a device implemented as a single component may include an adhesive interface to the subject including two or more electrodes that are applied to the subject. Adhesively applied stimulating devices embodied as a single component provide potential advantages such as easier application to the body of the subject, but may come at a disadvantage with regards to one or more of breathability, conformity, access to challenging interfaces, etc., relative to two component or multi-component arrangements.

A non-contacting stimulating device may be, for example an audio and/or visual system, a heating or cooling system, etc. Smart speakers and smart televisions or other displays are examples of audio and/or visual non-contacting stimulation devices. A smart speaker, for example, may be used to provide audible stimulus to the subject in the form of an alert, a suggestion, a command, music, other sounds, etc. Other examples of non-contacting stimulating devices include means for controlling temperature such as fans, air conditioners, heaters, etc.

One or more stimulating devices may also be incorporated in other systems, such as stimulating devices integrated into a bed, chair, operating table, exercise equipment, etc., that a subject interfaces with. A bed, for example, may include one or more pneumatic actuators, vibration actuators, shakers, or the like to provide a stimulus to the subject in response to a command, feedback signal or control signal generated based on measurement of one or more physiologic parameters of the subject utilizing one or more sensing devices.

Although the disclosure has discussed devices attached to the body for monitoring aspects of the subject's disorder and/or physiologic information, as well as providing a stimulus, therapeutic stimulus, etc., alternative devices may be considered. Non-contacting devices may be used to obtain movement information, audible information, skin blood flow changes (e.g., such as by monitoring subtle skin tone changes which correlate with heart rate), respiration (e.g., audible sounds and movement related to respiration), and the like. Such non-contacting devices may be used in place of or to supplement an on-body system for the monitoring of certain conditions, for applying stimulus, etc. Information captured by non-contacting devices may, on its own or in combination with information gathered from sensing devices on the body, be used to direct the application of stimulus to the subject, via one or more stimulating devices on the body and/or via one or more non-contacting stimulating devices.

In some embodiments, aspects of monitoring the subject utilizing sensing devices in the modular physiologic monitoring system may utilize sensing devices that are affixed to or embodied within one or more contact surfaces, such as surfaces on a piece of furniture on which a subject is positioned (e.g., the surface of a bed, a recliner, a car seat, etc.). The surface may be equipped with one or more sensors to monitor the movement, respiration, HR, etc., of the subject. To achieve reliable recordings, it is advantageous to have such surfaces be well positioned against the subject. It is also advantageous to build such surfaces to take into account comfort level of the subject to keep the subject from feeling the sensing surfaces and to maintain use of the sensing surface over time.

Stimulating devices, as discussed above, may take the form of audio, visual or audiovisual systems or devices in the sleep space of the subject. Examples of such stimulating devices include smart speakers. Such stimulating devices provide a means for instruction a subject to alter the sleep state thereof. The input or stimulus may take the form of a message, suggestion, command, audible alert, musical input, change in musical input, a visual alert, one or more lights, a combination of light and sound, etc. Examples of such non-contacting stimulating devices include systems such as Amazon Echo®, Google Home®, Apple Home Pod®, and the like.

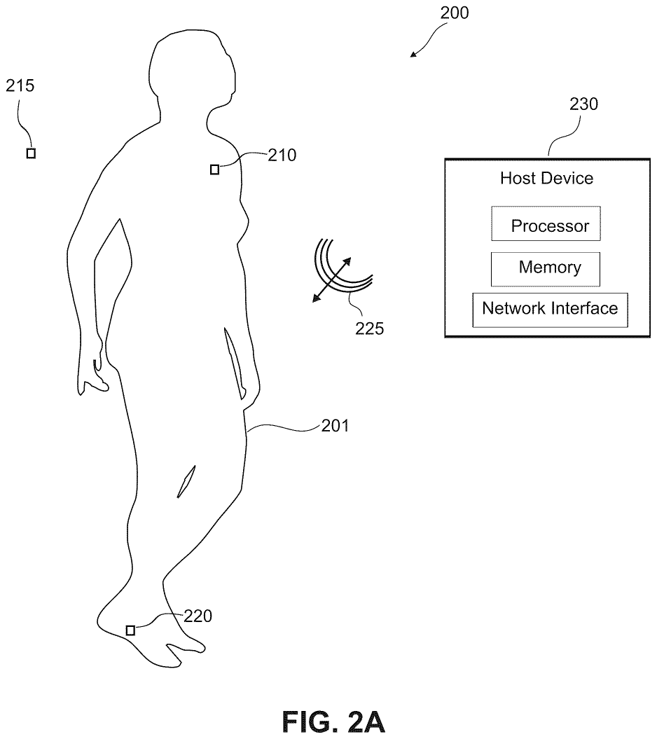

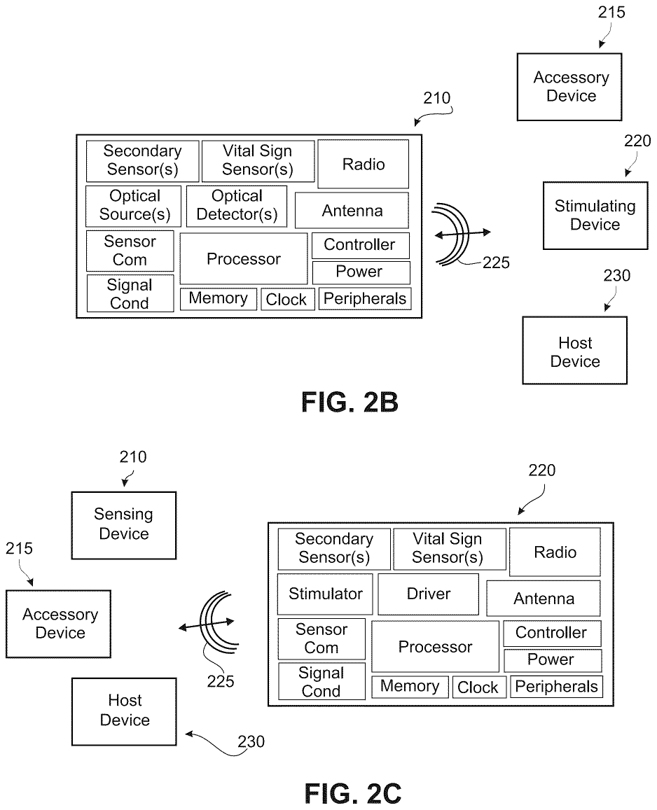

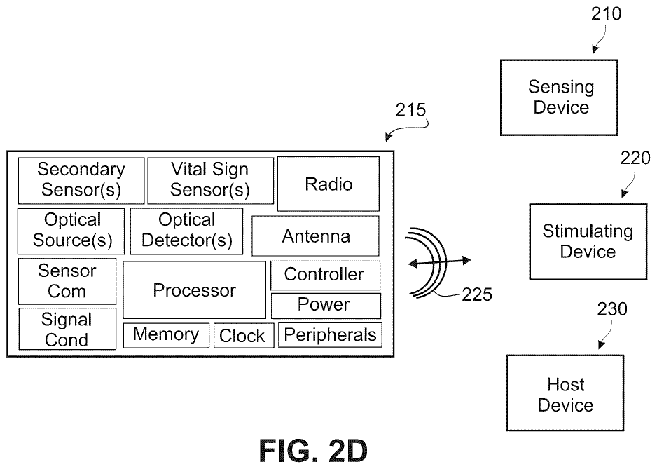

FIGS. 2A-2D show a modular physiologic monitoring system 200. The modular physiologic monitoring system 200 includes a sensing device 210, an accessory device 215 and a stimulating device 220 attached to a subject 201 that are in wireless communication 225 with a host device 230. The host device 230 includes a processor, a memory and a network interface.

The processor may comprise a microprocessor, a microcontroller, an application-specific integrated circuit (ASIC), a field-programmable gate array (FPGA) or other type of processing circuitry, as well as portions or combinations of such circuitry elements.

The memory may comprise random access memory (RAM), read-only memory (ROM) or other types of memory, in any combination. The memory and other memories disclosed herein may be viewed as examples of what are more generally referred to as “processor-readable storage media” storing executable computer program code or other types of software programs. Articles of manufacture comprising such processor-readable storage media are considered embodiments of the invention. A given such article of manufacture may comprise, for example, a storage device such as a storage disk, a storage array or an integrated circuit containing memory. The processor may load the computer program code from the memory and execute the code to provide the functionalities of the host device 230.

The network interface provides circuitry enabling wireless communication between the host device 230, the sensing device 210, the accessory device 215 and the stimulating device 220.

FIG. 2A illustrates a modular physiologic monitoring system 200 that includes only a single instance of the sensing device 210, the accessory device 215 and the stimulating device 220 for clarity. It is to be appreciated, however, that modular physiologic monitoring system 200 may include multiple sensing devices, accessory devices, and/or stimulating devices. In addition, although FIG. 2A illustrates a modular physiologic monitoring system 200 in which the sensing device 210 and the stimulating device 220 are attached to the subject 201 while the accessory device 215 is not attached to the subject 201, embodiments are not limited to such arrangements. As described above, one or more sensing and/or stimulating devices may be part of contacting surfaces or non-contacting devices. Further, accessory devices may alternatively be “on-body” or attached to the subject 201 as described elsewhere herein. In addition, the placement of sensing device 210 and stimulating device 220 on the subject 201 may vary as described above. Also, the host device 230 (and possible the accessory device 215) may be worn by the subject 201, such as being incorporated into a smartwatch or other wearable computing device. The functionality provided by host device 230 may also be provided, in some embodiments, by one or more of the sensing device 210, the accessory device 215 and the stimulating device 220. In some embodiments, as will be described in further detail below, the functionality of the host device 230 may be provided at least in part using cloud computing resources.

FIG. 2B shows a schematic diagram of aspects of the sensing device 210 in modular physiologic monitoring system 200. The sensing device 210 includes one or more of a processor, a memory device, a controller, a power supply, a power management and/or energy harvesting circuit, one or more peripherals, a clock, an antenna, a radio, a signal conditioning circuit, optical source(s), optical detector(s), a sensor communication circuit, vital sign sensor(s), and secondary sensor(s). The sensing device 210 is configured for wireless communication 225 with the accessory device 215, the stimulating device 220 and the host device 230.

FIG. 2C shows a schematic diagram of aspects of the stimulating device 220 in modular physiologic monitoring system 200. The stimulating device 220 includes one or more of a processor, a memory device, a controller, a power supply, a power management and/or energy harvesting circuit, one or more peripherals, a clock, an antenna, a radio, a signal conditioning circuit, a driver, a stimulator, vital sign sensor(s), a sensor communication circuit, and secondary sensor(s). The stimulating device 220 is configured for wireless communication 225 with the sensing device 210, the accessory device 215, and the host device 230.

FIG. 2D shows a schematic diagram of aspects of the accessory device 215 in modular physiologic monitoring system 200. The accessory device 215 includes one or more of a processor, a memory device, a controller, a power supply, a power management and/or energy harvesting circuit, one or more peripherals, a clock, an antenna, a radio, a signal conditioning circuit, a driver, a stimulator, vital sign sensor(s), a sensor communication circuit, and secondary sensor(s). The accessory device 215 is configured for wireless communication 225 with the sensing device 210, the stimulating device 220, and the host device 230.

Communication of data from the sensing devices and/or stimulating devices (e.g., patches and/or patch-module pairs), as well as accessory devices, may be performed via a local personal communication device (PCD). Such communication in some embodiments takes place in two parts: (1) local communication between a patch and/or patch-module pair (e.g., via a hub or module of a patch-module pair) and the PCD; and (2) remote communication from the PCD to a back-end server, which may be part of a cloud computing platform and implemented using one or more virtual machines (VMs) and/or software containers. The PCD and back-end server may collectively provide functionality of the host device as described elsewhere herein. The PCD may also be part of or provide functionality of an accessory device.

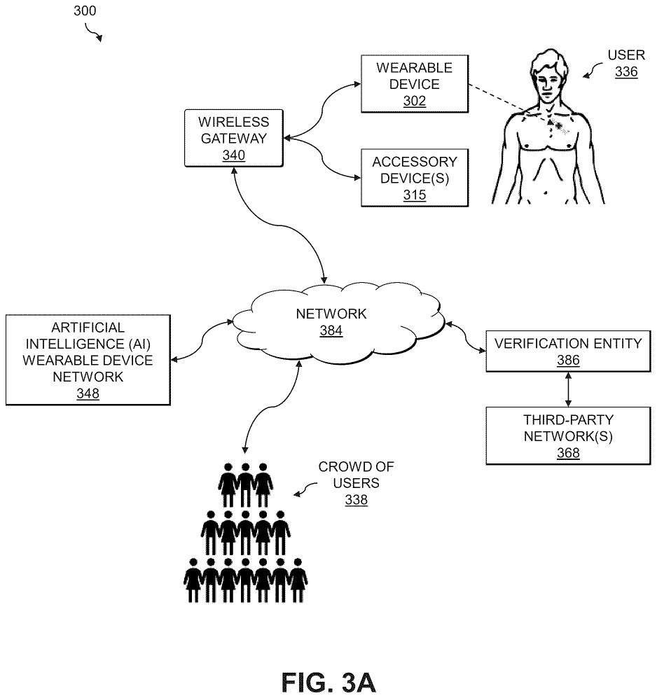

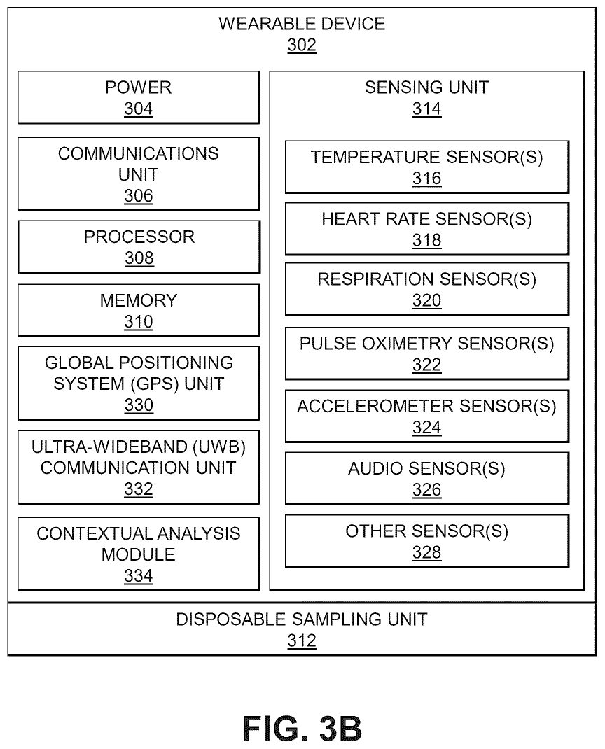

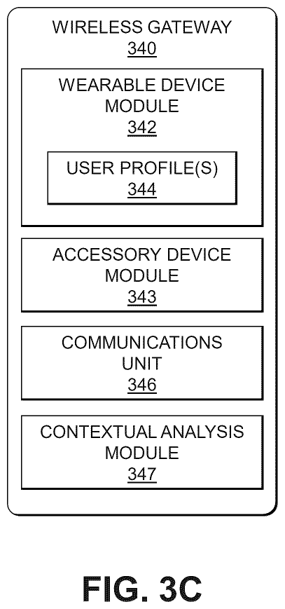

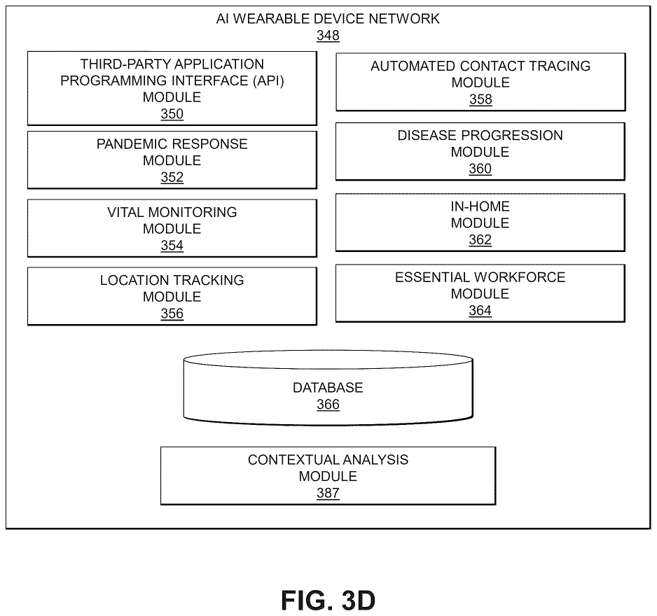

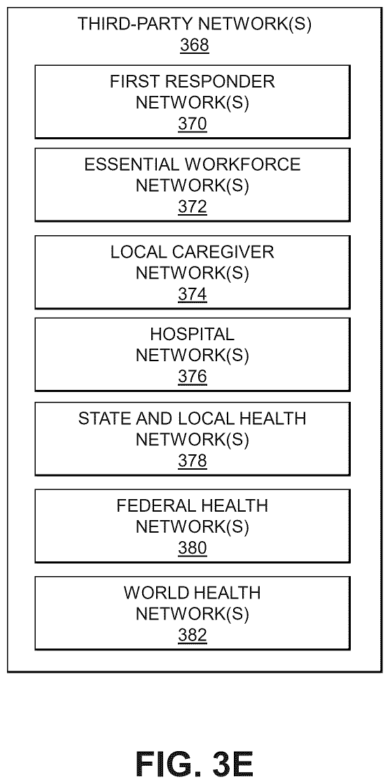

FIGS. 3A-3E show a wearable sensor system 300 configured for monitoring physiologic, location, and contextual and/or environmental data for a plurality of users, and for analyzing such data for use in health monitoring. The wearable sensor system 300 provides the capability for assessing the condition of the human body of a plurality of users (e.g., including user 336 and a crowd of users 338). As shown in FIG. 3A, the wearable sensor system 300 includes a wearable device 302 that is affixed to user 336, as well as one or more accessory devices 315 having sensors configured for capturing contextual and/or environmental information for the user 336 and/or the crowd of users 338. While the wearable device 302 is shown as being “on-body” relative to the user 336, the accessory devices 315 may, but are not required to be, “off-body” devices related to the user 336 and/or the crowd of users 338.

Data collected from the user 336 via the wearable device 302, as well as contextual and/or environmental data collected from the accessory devices 315, is communicated using a wireless gateway 340 to an artificial intelligence (AI) wearable device network 348 over or via network 384. The network 384 may comprise a physical connection (wired or wireless), the Internet, a cloud communication network, etc. Examples of wireless communication networks that may be utilized include networks that utilize Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. Also coupled to the network 384 is a crowd of users 338 and a verification entity 386 coupled to a set of third-party networks 368. Detailed views of the wearable device 302, wireless gateway 340, AI wearable device network 348 and third-party networks 368 are shown in FIGS. 3B-3E, respectively.

In some embodiments, the wearable device 302 is implemented using one or more patch-module pairs as described above with respect to FIGS. 1 and 2A-2C. The patch-module pairs described above with respect to FIGS. 1 and 2A-2C, however, are just one example of wearable technology that may be used to provide the wearable device 302. Various other types of wearable technology may be used to provide the wearable device in other embodiments, including but not limited to wearables, fashion technology, tech togs and other types of fashion electronics that include “smart” electronic devices (e.g., electronic devices with micro-controllers) that can be incorporated into clothing or worn on the body as implants or accessories. Wearable devices such as activity trackers are examples of Internet of Things (IoT) devices, and such “things” include electronics, software, sensors and connectivity units that are effectors enabling objects to exchange data (including data quality) through the Internet with a manufacturer, operator and/or other connected devices without requiring human intervention. Wearable technology has a variety of applications, which grows as the field itself expands. Wearable technology appears prominently in consumer electronics with the popularization of smartwatches and activity trackers. Apart from commercial uses, wearable technology is being incorporated into navigation systems, advanced textiles, and health care.

In some embodiments, the wearable device 302 is capable of detecting and collecting medical data (e.g., body temperature, respiration, heart rate, etc.) from the wearer (e.g., user 336). The wearable device 302 can remotely collect and transmit real-time physiological data to health care providers and other caretakers responsible for ensuring their communities stay healthy. The wearable sensor system 300, in some embodiments, is user-friendly, hypoallergenic, unobtrusive, and cost-effective. In service of enabling remote evaluation of individual health indicators, the wearable sensor system 300 is configured to transmit data directly into existing health informatics and health care management systems from the comfort of patients' homes. The wearable device 302 is designed to monitor the cardiopulmonary state of a subject (e.g., user 336) over time in home or in clinical settings. Onboard sensors of the wearable device 302 can quantitatively detect and track severity of a variety of disease symptoms including fever, coughing, sneezing, vomiting, infirmity, tremor, and dizziness, as well as signs of decreased physical performance and changes in respiratory rate/depth. The wearable device 302 may also have the capability to monitor blood oxygenation.

In some embodiments, the wearable device 302 collects physiologic monitoring data from the subject user 336 utilizing a combination of a disposable sampling unit 312 and a reusable sensing unit 314 (FIG. 3B). The patch-module pairs described above with respect to FIGS. 1 and 2A-2C are an example implementation of the disposable sampling unit 312 and reusable sensing unit 314. The disposable sampling unit 312 may be formed from a softer-than-skin patch. The wearable device 302, formed from the combination of the disposable sampling unit 312 and reusable sensing unit 314, is illustratively robust enough for military use, yet extremely thin and lightweight. For example, the disposable sampling unit 312 and reusable sensing unit 314 may collectively weigh less than 0.1 ounce, about the same as a U.S. penny. The wearable device 302 may be adapted for placement almost anywhere on the body of the user 336, such as the various placement sites shown in FIG. 1 and described above.

In addition to the disposable sampling unit 312 and reusable sensing unit 314, the wearable device 302 may include a number of other components as illustrated in FIG. 3B. Such components include a power source 304, a communications unit 306, a processor 308, a memory 310, a GPS unit 330, an UWB communication unit 332, and radio switching logic 334.

The power source or component 304 of the wearable device 302, in some embodiments, includes one or more modules with each module including a power source (e.g., a battery, a rechargeable battery, an energy harvesting transducer, a microcircuit, an energy reservoir, a thermal gradient harvesting transducer, a kinetic energy harvesting transducer, a radio frequency energy harvesting transducer, a fuel cell, a biofuel cell, combinations thereof, etc.).

The communications unit 306 of the wearable device 302 may be embodied as communication circuitry, or any communication hardware that is capable of transmitting an analog or digital signal over one or more wired or wireless interfaces. In some embodiments, the communications unit 306 includes transceivers or other hardware for communications protocols, such as Near Field Communication (NFC), WiFi, Bluetooth, infrared (IR), modem, cellular, ZigBee, a Body Area Network (BAN), and other types of wireless communications. The communications unit 306 may also or alternatively include wired communication hardware, such as one or more universal serial bus (USB) interfaces.

The processor 308 of the wearable device 302 is configured to decode and execute any instructions received from one or more other electronic devices and/or servers. The processor 308 may include any combination of one or more general-purpose processors (e.g., Intel® or Advanced Micro Devices (AMD)® microprocessors), one or more special-purpose processors (e.g., digital signal processors or Xilink® system on chip (SOC) field programmable gate array (FPGA) processors, application-specific integrated circuits (ASICs), etc.), etc. The processor 308 is configured in some embodiments to execute one or more computer-readable program instructions, such as program instructions to carry out any of the functions described herein including but not limited to those of the contextual analysis module 334 described below. The processor 308 is illustratively coupled to the memory 310, with the memory 310 storing such computer-readable program instructions.

The memory 310 may include, but is not limited to, fixed hard disk drives, magnetic tape, floppy diskettes, optical disks, compact disc read-only memories (CD-ROMs), magneto-optical disks, semiconductor memories such as read-only memory (ROM), random-access memory (RAM), programmable ROM (PROM), erasable PROM (EPROM), electrically erasable PROM (EEPROM), flash memory, magnetic or optical cards, or other type of media/machine-readable medium suitable for storing electronic instructions. The memory 310 may comprise modules implemented as one or more programs. In some embodiments, a non-transitory processor-readable storage medium has stored therein program code of one or more software programs, wherein the program code when executed by at least one processing device (e.g., the processor 308) causes said at least one processing device to perform one or more aspects of the methods, algorithms and process flows described herein.

The processor 308 and memory 310 are an example of a processing device or controller. The controller may comprise a central processing unit (CPU) for carrying out instructions of one or more computer programs for performing arithmetic, logic, control and input/output (I/O) operations specified by the instructions (e.g., as specified by the contextual analysis module 334 as described in further detail below). Such computer programs may be stored in the memory 310. The memory 310 provides electronic circuitry configured to temporarily store data that is utilized by the processor 308. In some embodiments, the memory 310 further provides persistent storage for storing data utilized by the processor 308. Although not explicitly shown, other components of the wearable sensor system 300 (e.g., the accessory devices 315, the wireless gateway 340 (FIG. 3C), the AI wearable device network 348, one or more of the third-party networks 368, the verification entity 386, etc.) may also include one or more processors coupled to one or more memories providing processing devices implementing the functionality of such components.

As noted above, the wearable device 302 illustratively includes the disposable sampling unit 312 which may be embodied as a physical interface to the skin of the user 336. Patches as described elsewhere herein are examples of a disposable sampling unit 312. Such patches are adapted for attachment to a human or animal body (e.g., attachable to the skin thereof, reversibly attachable, adhesively attachable, with a disposable interface that couples to a reusable module, etc.). In some embodiments, the disposable sampling unit 312 is part of a system that is capable of modular design, such that various wearable devices or portions thereof (e.g., reusable sensing unit 314) are compatible with various disposable sampling units with differing capabilities. In some embodiments, the patch or more generally the disposable sampling unit 312 allows sterile contact between the user 336 and other portions of the wearable device 302, such as the reusable sensing unit 314. In such embodiments, the other portions of the wearable device 302 (e.g., which may be embodied as a module as described above with respect to FIGS. 1 and 2A-2C) may be returned, sterilized and reused (e.g., by the same user 336 or another user) while the patch or disposable sampling unit 312 is disposed of. In some embodiments, the patch or other disposable sampling unit 312 is suitable for wearing over a duration of time in which the user 336 is undergoing physiological monitoring. In such embodiments, the patch or disposable sampling unit 312 may be disposed of after the monitoring duration has ended.

The reusable sensing unit 314 includes various sensors, such as one or more temperature sensors 316, one or more heart rate sensors 318, one or more respiration sensors 320, one or more pulse oximetry sensors 322, one or more accelerometer sensors 324, one or more audio sensors 326, and one or more other sensors 328. One or more of the sensors 316-328 may be embodied as electric features, capacitive elements, resistive elements, touch sensitive components, analyte sensing elements, printed electrochemical sensors, light sensitive sensing elements, electrodes (e.g., including but not limited to needle electrodes, ionically conducting electrodes, reference electrodes, etc.), electrical traces and/or interconnects, stretch sensing elements, contact interfaces, conduits, microfluidic channels, antennas, stretch resistant features, stretch vulnerable features (e.g., a feature that changes properties reversibly or irreversibly with stretch), strain sensing elements, photo-emitters, photodiodes, biasing features, bumps, touch sensors, pressure sensing elements, interfacial pressure sensing elements, piezoelectric elements, piezoresistive elements, chemical sensing elements, electrochemical cells, electrochemical sensors, redox reactive sensing electrodes, light sensitive structures, moisture sensitive structures, pressure sensitive structures, magnetic structures, bioadhesives, antennas, transistors, integrated circuits, transceivers, sacrificial structures, water soluble structures, temperature sensitive structures, light sensitive structures, light degrading structures, flexible light emitting elements, piezoresistive elements, moisture sensitive elements, mass transfer altering elements, etc.

In some embodiments, one or more of the sensors 316-328 have a controlled mass transfer property, such as a controlled moisture vapor conductivity so as to allow for a differential heat flux measurement through the patch or other disposable sampling unit 312. Such properties of one or more of the sensors 316-328 may be used in conjunction with the one or more temperature sensors 316 to obtain core temperature measurements of the user 336. It should be noted that one or more of the sensors 316-328 or the sensing unit 314 generally may be associated with signal conditioning circuitry used in obtaining core temperature or other measurements of physiologic parameters of the user 336. Core temperature measurements may, in some embodiments, be based at least in part on correlation parameters extracted from sensors of multiple wearable devices, or from sensors of the same wearable device that interface with different portions of the user 336. The correlation parameters may be based on thermal gradients computed as comparisons of multiple sensor readings (e.g., from a first subset of sensors oriented to make thermal contact with the user 336 and from a second subset of sensors oriented to make thermal contact with ambient surroundings, etc.). Core temperature readings may thus be estimated from the thermal gradients.

Changes in core temperature readings from multiple sensor readings over some designated period of time (e.g., a transitionary period where two wearable devices are attached to the user 336 and obtain core temperature readings) are analyzed to generate correlation parameters that relate changes in core temperature readings from the multiple sensors. In some embodiments, this analysis includes determining which of the multiple sensors has a lowest thermal gradient and weighting the correlation parameters to the sensor or device having the lowest thermal gradient. Consider an example where a first set of one or more sensors is at a first site on the user 336 and a second set of one or more sensors is at a second site on the user 336, with the first site being associated with a lower thermal gradient than the second site but with the second site being more conducive to long-term wear relative to the first site. In such cases, it may be desired to obtain core temperature readings from the first and second sets of sensors, establish the correlation parameter, and then subsequently use only the second set of sensors at the second site more conducive to long-term wear by the user 336. In some embodiments, the temperature sensors 316 comprise one or more digital infrared temperature sensors (e.g., Texas Instruments TMP006 sensors).

The heart rate sensors 318 in some embodiments are configured to sense physiological parameters of the user 336, such as conditions of the cardiovascular system of the user 336 (e.g., heart rate, blood pressure, heart rate variability, etc.). In some embodiments, the physiological parameters comprise one or more bioimpedance measurements, and correlation parameters may be generated by extracting local measures of water content from bioimpedance signals recorded from multiple sensors potentially at different sites on the body of the user 336. The local measures of water content recorded by different devices or sensors may be recorded during at least a portion of a transitionary period as described above to generate correlation parameters for application to bioimpedance signals recorded by the different sensors to offset at least a portion of identified differences therebetween. The correlated changes in the local measures of water content may be associated with a series of postural changes by the user 336.

The respiration sensors 320 are configured to monitor the condition of respiration, rate of respiration, depth of respiration, and other aspects of the respiration of the user 336. The respiration sensors 320 may obtain such physiological parameters by placing the wearable device 302 (e.g., a patch-module pair thereof) on the abdomen of the user 336 for monitoring movement or breathing, below the rib cage for monitoring respiration (generally on the right side of the body to substantially reduce EKG influences on the measurements), such placement enabling the respiration sensors 320 to provide rich data for respiration health, which may be advantageous in detection of certain infectious diseases that affect the respiratory tract of victims, such as, for example, coronavirus/COVID-19.

The pulse oximetry sensors 322 are configured to determine oxygen saturation (SpO2) using a pulse oximeter to measure the oxygen level or oxygen saturation of the blood of the user 336.

The accelerometer sensors 324 are configured to measure acceleration of the user 336. Single and multi-axis models of accelerometers may be used to detect the magnitude and direction of the proper acceleration as a vector quantity, and can be used to sense orientation (e.g., based on the direction of weight changes), coordinate acceleration, vibration, shock, and falling in a resistive medium (e.g., a case where the proper acceleration changes, since it starts at zero then increases). The accelerometer sensors 324 may be embodied as micromachined microelectromechanical systems (MEMS) accelerometers present in portable electronic devices such as the wearable device 302. The accelerometer sensors 324 may also be used for sensing muscle contraction for various activities, such as running and other erect sports. In the case of running and other erect sports, resistance rises as either (or both) of the right and left extremities (e.g., feet, shins, knees, etc.) strike the ground. This rise or peak may be synchronized to bolus ejection as detailed herein. The accelerometer sensors 324 may detect such activity by measuring the body or extremity center of mass of the user 336. In some cases, the body center of mass may yield the best timing for the injection of fluid. Embodiments, however, are not limited solely to use with measuring the body center of mass.

The audio sensors 326 are configured to convert sound into electrical signals, and may be embodied as one or more microphones or piezoelectric sensors that use the piezoelectric effect to measure changes in pressure, acceleration, temperature, strain, or force by converting them to an electrical charge. In some embodiments, the audio sensors 326 may include ultrasonic transducer receivers capable of converting ultrasound into electrical signals.

It should be noted that the sensors 316-326 described above are presented by way of example only, and that the sensing unit 314 may utilize various other types of sensors 328 as described elsewhere herein. For example, in some embodiments the other sensors 328 include one or more of motion sensors, humidity sensors, cameras, radiofrequency receivers, thermal imagers, radar devices, lidar devices, ultrasound devices, speakers, etc.

The GPS unit 330 is a component of the wearable device 302 configured to detect global position using GPS, a satellite-based radio navigation system owned by the U.S. government and operated by the U.S. Space Force. GPS is one type of global navigation satellite system (GNSS) that provides geolocation and time information to a GPS receiver anywhere on or near the Earth where there is an unobstructed line of sight to four or more GPS satellites.

The UWB communication unit 332 is a component of the wearable device 302 configured to detect UWB radiofrequencies. UWB is a short-range, wireless communication protocol similar to Bluetooth or WiFi, which uses radio waves at a very high frequency. Notably, UWB also uses a wide spectrum of several gigahertz (GHz). The functioning of a UWB sensor is to provide the ability to continuously scan an entire room and provide spatial awareness data to the wearable device 302, improving the localization of the wearable device 302 particularly in conjunction with use of the GPS unit 330.

The contextual analysis module 334 is configured to execute various functionality for combining sensor data from the sensing unit 314 (e.g., physiologic monitoring data for the user 336) along with sensor data from the accessory devices 315 (e.g., contextual and/or environmental information associated with the user 336 and/or the crowd of users 338) for higher-level analysis.

The user 336 may be a human or animal to which the wearable device 302 is attached. Sensor data and localization data collected by the wearable device 302, along with contextual and/or environmental data collected from the accessory devices 315, may be provided to AI wearable device network 348 for analysis, with portions or such analysis being provided to one or more of the third-party networks 368 for various purposes. Communication of the sensor and localization data from the wearable device 302, as well as communication of the contextual and/or environmental data from the accessory devices 315, to the AI wearable device network 348 may take place via a wireless gateway 340, with the communication between the wireless gateway 340 and the AI wearable device network 348 taking place over one or more networks 384.

As shown in FIG. 3C, the user 336 may configure the wireless gateway 340 to include a user profile 344. The user profile 344 may include various health and physiological data about the user 336 that may not be obtained by sensors 316-328 of the wearable device 302. The user profile 344, for example, may include information such as a name (e.g., first, last and middle name), biological sex, age (e.g., in years), weight (e.g., in pounds, kilograms, etc.), and height (e.g., in feet or inches, in meters, etc.). The user profile 344 may also include known diseases and disorders (e.g., asthma, allergies, current medications, family medical history, other medical data, etc.), where such information may include Protected Health Information (PHI) regulated by American Health Insurance Portability and Accountability Act (HIPAA) or other applicable rules and regulations. PHI includes individually identifiable health information that relates to one or more of: the past, present, or future physical or mental health or condition of an individual; provision of health care to the individual by a covered entity (e.g., a hospital or doctor); the past, present, or future payment for the provision of health care to the individual; telephone numbers, fax numbers, email addresses, Social Security numbers, medical record numbers, health plan beneficiary numbers, license plate numbers, uniform resource locators (URLs), full-face photographic images or any other unique identifying numbers, characteristics, codes, or combination thereof that allows identification of an individual. The user profile 344 may further include an emergency contact (e.g., name, phone number, address, etc.), next of kin (e.g., name, phone number, address, etc.), preferred hospital (e.g., name, phone number, address, etc.) and primary care physician (PCP) of the user 336 (e.g., name, phone number, place of business, etc.). The user profile 344 may further include local caregiver information (e.g., name, phone number, address, etc.) and preferred first responder network information (e.g., name, phone number, address, etc.). The local caregiver may be, for example, a nursing agency, a private caregiver such as a family member, a nursing home, or other local caregivers such as physical therapists, chiropractors, pharmacists, pediatricians, acupuncture specialists, massage therapists, etc. In some cases, the local caregiver is associated with one or more telemedicine networks. The preferred first responder network may be, for example, a local hospital and/or a local ambulatory rescue agency. In some embodiments, the preferred first responder network may be an interface with an emergency calling network (e.g., 911).

The wireless gateway 340 sends the sensor data and localization data obtained from the user 336 by the wearable device 302, as well as contextual and/or environmental data obtained from the accessory devices 315, utilizing communications unit 346, which may comprise any type of transceiver for coupling the wireless gateway 340 to the network 384. The communications unit 346 of the wireless gateway 340 may be embodied as communication circuitry or any communication hardware capable of transmitting an analog or digital signal over wired or wireless network interfaces. Such network interfaces may support not only communication with the AI wearable device network 348 over network 384, but also communications between the wearable device 302 and the wireless gateway 340. Any combination of network types may be utilized, including but not limited to UWB, NFC, WiFi, Bluetooth, BLE, IR, modem, cellular, ZigBee, BAN, etc. The wireless gateway 340 may also be provisioned with contextual analysis module 347, which provides functionality similar to that of the contextual analysis module 334.

The wireless gateway 340 may be, for example, a smartphone, a tablet, a laptop or desktop computer, an Internet-connected modem, a wireless router or standalone wireless hub device connected to the Internet, etc. The wireless gateway 340, in some embodiments, may itself comprise or be incorporated into one or more wearable devices (e.g., a smartwatch, an activity tracker, etc.). In some cases, the wireless gateway 340 may be part of the wearable device 302, or vice versa. The wireless gateway 340 is illustratively a smart device that is owned or controlled by the user 336, such as a smartphone, and allows rapid onboarding of wearable devices such as wearable device 302 to the AI wearable device network 348.

The wireless gateway 340 includes a wearable device module 342 and accessory device module 343 that provides software programs or computer instructions for providing various functionality of the wireless gateway 340. Although not shown in FIG. 3C, the wireless gateway 340 is assumed to comprise at least one processing device or controller including a processor coupled to a memory for executing the functionality of the wearable device module 342 and the accessory device module 343. Such functionality may include, for example, wirelessly pairing the wearable device 302 and one or more of the accessory devices 315 in a BAN associated with the user 336. Such functionality may also include receiving the sensor data and the localization data from the wearable device 302 and the contextual and/or environmental data from the accessory devices 315 via the communications unit 346, and possibly performing a preliminary analysis of the sensor data, the localization data and the contextual and/or environmental data. Such analysis may be based at least in part on information stored in the user profile 344. Based on such analysis, the wearable device module 342 and the accessory devices module 343 may determine whether any immediate notifications should be provided to the user 336. Such notifications may comprise, for example, indications of symptoms associated with at least one disease state. In other embodiments, the wearable device 302 functions as a pass-through entity and does not perform such preliminary analysis. Instead, the wireless gateway 340 may provide the sensor data and the localization data received from the wearable device 302, along with the associated user profile 344 and the contextual and/or environmental data obtained from the accessory devices 315, to the AI wearable device network 348 over network 384 as a pass-through entity.

Regardless of whether or not the wireless gateway 340 performs such preliminary analysis, the wearable device module 342 and the accessory device module 343 of the wireless gateway 340 may receive any combination of diagnostic information, world health information, sensor data analysis, localization analysis, analysis created from a fusion of data from a plurality of sensors from the AI wearable device network 348, etc. At least a portion of the received information is based on analysis of the sensor data, the localization data, the user profile 344, the contextual and/or environmental data, or information derived therefrom previously provided by the wireless gateway 340 to the AI wearable device network 348. At least a portion of the received information is used to generate notifications or other output via a graphical user interface (GUI) of the wireless gateway 340, the wearable device 302, one or more of the accessory devices 315, or another type of local or remote indicator device.

The wearable device module 342 and/or the accessory device module 343 may provide functionality for determining notification settings associated with the user 336, and to execute or deliver notifications in accordance with the determined notification settings utilizing the wearable device 302 and/or one or more of the accessory devices 315 or other devices. The notification settings, in some embodiments, may specify the types of indicator devices that are part of or otherwise accessible to the wearable device 302 and/or the accessory devices 315 for delivering notifications to the user 336 (or to a doctor, nurse, physical therapist, medical assistant, caregiver, etc. associated with the user 336). The indicator devices in some embodiments may be configured to deliver visual or audible alarms. In other embodiments, the indicator devices may be configured to provide stimulus or feedback via stimulating devices as described elsewhere herein. Such stimulus or feedback, as detailed above, may include physical stimulus (e.g., electrical, thermal, vibrational, pressure, stroking, a combination thereof, or the like), optical stimulus, acoustic stimulus, etc. In some embodiments, notifications may be delivered to remote terminals or devices other than the wearable device 302 and/or the accessory devices 315 associated with user 336. For example, notifications may be delivered to one or more devices associated with a doctor, nurse, physical therapist, medical assistant, caregiver, etc. associated with the user 336.

The notification delivery method may also or alternatively comprise a visual or audible read-out or alert from a “local” device that is in communication with the wearable device 302. The local device may comprise, for example, a mobile computing device such as a smartphone, tablet, laptop etc., or another computing device, that is associated with the user 336. The wearable device 302 is one example of a local device. A local device may also include devices connected to the wearable device 302 via a BAN or other type of local or short-range wireless network (e.g., a Bluetooth network connection).

The notification delivery method may further or alternatively comprise a visual or audible read-out or alert from a “remote” device that is in communication with the wearable device 302 or the wireless gateway 340 via network 384, such as one or more of the accessory devices 315. The remote device may be a mobile computing device such as a smartphone, tablet, laptop, etc., or another computing device (e.g., a telemetry center or unit within a hospital or other facility), that is associated with a doctor, nurse, physical therapist, medical assistant, caregiver, etc. monitoring the user 336. It should be understood that the term “remote” in this context does not necessarily indicate any particular physical distance from the user 336. For example, a remote device to which notifications are delivered may be in the same room as the user 336. The term “remote” in this context is instead used to distinguish from “local” devices (e.g., in that a “local” device in some embodiments is assumed to be owned by, under the control of, or otherwise associated with the user 336, while a “remote” device is assumed to be owned by, under the control of, or otherwise associated with a user or users other than the user 336 such as a doctor, nurse, physical therapist, medical assistance, caregiver, etc.).

The indicator devices may include various types of devices for delivering notifications to the user 336 (or to a doctor, nurse, physical therapist, medical assistant, caregiver, etc. associated with the user 336). In some embodiments, one or more of the indicator devices comprise one or more light emitting diodes (LEDs), a liquid crystal display (LCD), a buzzer, a speaker, a bell, etc., for delivering one or more visible or audible notifications. More generally, the indicator devices may include any type of stimulating device as described herein which may be used to deliver notifications to the user 336 (or to a doctor, nurse, physical therapist, medical assistant, caregiver, etc. associated with the user 336).

FIG. 3A also shows the crowd of users 338, each of which is assumed to provide sensor data and localization data obtained by a plurality of wearable devices to the AI wearable device network 348, possibly via respective wireless gateways. The wearable devices and wireless gateways for the crowd of users 338 may be configured in a manner similar to that described herein with respect to the wearable device 302 and wireless gateway 340 associated with the user 336.

It should be appreciated that although the wearable device 302 may be configured with multiple different types of sensors 316-328, it is generally not possible to configure a wearable device with every possible sensor that may be needed in different scenarios. For example, wearable devices are advantageously designed for comfortable wear and use, and thus may require a small form factor which cannot accommodate the possible range of sensors and sensor types which may be needed in different scenarios. Further, some types of sensors are large, heavy and/or expensive, and thus are not conducive to being incorporated as part of a wearable device. Nonetheless, different tasks may benefit from the use of contextual and/or environmental information which may be provided using sensor types that are not available in the wearable device 302.

Consider, as an example, a scenario in which the user 336 is placed in an environment with possible radiation exposure where dosimeter sensors would be advantageous (e.g., for correlating changes in physiologic parameters obtained from the sensing unit 314 of the wearable device 302 with knowledge of an amount and/or type of radiation that the user 336 is exposed to). The wearable device 302 may not be configured with dosimeter sensors, as this may not be practical (e.g., due to the size, power, material and other requirements) or such a potential use case is not expected to come up very often. When the need arises for radiation exposure information, accessory sensing devices 315 that include dosimeter sensors may be leveraged to provide such information which is used for contextual analysis (e.g., implemented by the contextual analysis module 334 on the wearable device 302, on the contextual analysis module 347 of the wireless gateway 340, on the contextual analysis module 387 of the Al wearable device network 348, on contextual analysis modules implemented by the accessory devices 315 and/or one or more of the third-party networks 368, etc.).

The accessory sensing devices 315 may also or alternatively be used to determine the user 336's microenvironmental exposure to light, noise, temperature, humidity, pressure, etc. These and other factors can influence different aspects of the microenvironment of the user 336 which can be correlated with physiologic data obtained from the user 336 via the sensing unit 314 of the wearable device 302. This may include use cases such as impact/fall detection, detecting fatigue of the user 336, etc. Another use case is in determining a “wet-bulb” temperature of the user 336. The wet-bulb temperature of the user 336, which may be determined from microenvironmental monitoring of information such as light, temperature, humidity and pressure, can be correlated with measured physiologic data to determine harmful and potentially life-threatening conditions. Monitoring for the microenvironmental wet-bulb temperature can be useful in various scenarios, including for soldiers which may be equipped with Mission Oriented Protective Posture (MOPP) gear that is heavy and bulky, leading to the soldiers having an increased wet-bulb temperature. The microenvironmental monitoring of the wet-bulb temperature (e.g., via accessory devices 315) may be correlated with physiologic data measured from on-body sensors (e.g., from the sensing unit 314 of the wearable device 302) which characterize, for example, physical activity or exertion. This may be used to provide feedback to the user 336 (e.g., to stop the physical activity or exertion, to remove MOPP gear or other bulky equipment or clothing, etc.). In some embodiments, the microenvironmental monitoring of wet-bulb temperature may be correlated with local weather report information as well as measured physiologic data of the user 336 (e.g., to detect risk of heat exhaustion or other conditions).

The microenvironmental monitoring may also or alternatively utilize microenvironmental noise information to detect exposure to potentially harmful noise levels. This may include monitoring and detecting a microenvironmental infrasound signature, which may be correlated with physiologic data from the user 336 to characterize effects such as nausea, vomiting internal injuries (e.g., organ tearing), etc. Noise exposure information may also be used to detect microenvironmental sound signatures (e.g., to detect exposure to radiofrequency (RF), to detect drones or vehicles in the area, to detect exposure to shots fired/explosions, to detect sounds indicative of coughing, vomiting or choking events, etc.) which may be time-correlated with physiologic data from the user 336 (e.g., core vital signs indicative of being hit by a shot fired, having injuries related to a blast exposure, being sick from dehydration, vomiting, choking, etc.).

The microenvironmental information and physiologic monitoring data may be used for various types of contextual analysis, where the microenvironmental information and physiologic monitoring data are correlated with knowledge of what the user 336 is doing (e.g., whether the user 336 is awake or asleep, a physical workload or profile of the user 336, etc.). Noise information, in some cases, may be used for contextual analysis of the activity of multiple users (e.g., the user 336 and one or more of the users in the crowd of users 338) to provide spatial reference information (e.g., detecting where shots/blasts come from, where drones or vehicles are traveling, etc.). In some cases, the contextual analysis includes “friend/foe” detection, where the user 336 has a specific profile (e.g., ECG signature, tone/audio signature, etc.) which may be used to detect when the wearable device 302 associated with the user 336 is being utilized by another user (e.g., a potential “foe”).

More generally, the accessory sensing devices 315 are leveraged to provide contextual and/or environmental information which is difficult, not possible or not practical to obtain utilizing the wearable device 302 alone. This may be due to the contextual and/or environmental information only being needed in limited use cases, such that the cost of implementing the required sensor types within the wearable device 302 is not practical or cost-effective. Thus, it should be appreciated that the sensor types of the accessory devices 315 which are leveraged to obtain contextual and/or environmental information are not limited solely to sensor types which are difficult to implement within the small form factor other constraints of the wearable device 302 (e.g., comfortable long-term wear by the user 336, cost, etc.).

The AI wearable device network 348 is configured to receive data (e.g., sensor data and localization data from the wearable device 302, contextual and/or environmental data from the accessory devices 315, user profile 344, preliminary analysis of the sensor, localization and contextual and/or environmental data, etc.) from the wireless gateway 340 and the crowd of users 338. The AI wearable device network 348 analyzes the received data using various software modules implementing AI algorithms for determining disease states, types of symptoms, risk of infection, contact between users, condition of physiological parameters, occurrence of events, event classification, etc. As shown in FIG. 3D, such modules include a third-party application programming interface (API) module 350, a pandemic response module 352, a vital monitoring module 354, a location tracking module 356, an automated contact tracing module 358, a disease progression module 360, an in-home module 362 and an essential workforce module 364. The AI wearable device network 348 also includes a database 366 configured to store the received data, results of analysis on the received data, data obtained from third-party networks 368, etc. The AI wearable device network 348 further implements contextual analysis module 387 configured to provide functionality similar to that of the contextual analysis module 334.

In some embodiments, the AI wearable device network 348 is implemented as an application or applications running on one or more physical or virtual computing resources. Physical computing resources include, but are not limited to, smartphones, laptops, tablets, desktops, wearable computing devices, servers, etc. Virtual computing resources include, but are not limited to, VMs, software containers, etc. The physical and/or virtual computing resources implementing the AI wearable device network 348, or portions thereof, may be part of a cloud computing platform. A cloud computing platform includes one or more clouds providing a scalable network of computing resources (e.g., including one or more servers and databases). In some embodiments, the clouds of the cloud computing platform implementing the AI wearable device network 348 are accessible via the Internet over network 384. In other embodiments, the clouds of the cloud computing platform implementing the AI wearable device network 348 may be private clouds where access is restricted (e.g., such as to one or more credentialed medical professionals or other authorized users). In these and other embodiments, the AI wearable device network 348 may be considered as forming part of an emergency health network comprising at least one server and at least one database (e.g., the database 366) storing health data pertaining to a plurality of users (e.g., the user 336 and crowd of users 338).

The database 366 provides a data store for information about patient conditions (e.g., information about the user 336 and crowd of users 338), information relating to diseases including epidemics or pandemics, etc. Although shown as being implemented internal to the AI wearable device network 348 in FIG. 3D, it should be appreciated that the database 366 may also be implemented at least in part external to the AI wearable device network 348 (e.g., as a standalone server or storage system). The database 366 may be implemented as part of the same cloud computing platform that implements the AI wearable device network 348.

The AI wearable device network 348 may exchange various information with third-party network 368. As shown in FIG. 3E, the third-party network 368 may include any combination of one or more first responder networks 370, one or more essential workforce networks 372, one or more local caregiver networks 374, one or more hospital networks 376, one or more state and local health networks 378, one or more federal health networks 380, one or more world health networks 382, etc. Third-party networks 368 may also include telemedicine networks. For example, in some embodiments one or more of the local caregiver networks 374 may comprise or be associated with one or more telemedicine networks, such that local caregivers of the local caregiver networks 374 may provide care to patients or users via telemedical communications. Under certain circumstances, as permitted by the verification entity 386, one or more of the third-party networks 368 may receive data and analysis from the AI wearable device network 348, for various purposes including but not limited to diagnosis, instruction, pandemic monitoring, disaster response, resource allocation, medical triage, any other tracking or intervention and associated logistics, etc. The first responder networks 370 may include any person or team with specialized training who is among the first to arrive and provide assistance at the scene of an emergency, such as an accident, natural disaster, terrorism, etc. First responders include, but are not limited to, paramedics, emergency medical technicians (EMTs), police officers, fire fighters, etc. The essential workforce networks 372 may include networks for employers and employees of essential workforces of any company or government organization that continues operation during times of crises, such as a viral pandemic. Essential workforces include, but are not limited to, police, medical staff, grocery workers, pharmacy workers, other health and safety service workers, etc. The local caregiver networks 374 may include a network of local clinics, family doctors, pediatricians, in-home nurses, nursing home staff, and other local caregivers. The hospital networks 376 allow transfer of data between hospitals and the AI wearable device network 348.

The exchange of information between the AI wearable device network 348 and third-party networks 368 may involve use of a verification entity 386, which ensures data security in accordance with applicable rules and regulations (e.g., HIPAA). The AI wearable device network 348 utilizes the third-party API module 350 to perform such verification of the third-party networks 368 utilizing the verification entity 386, before providing any data or analysis thereof related to the user 336 or crowd of users 338 to any of the third-party networks 368. It should be noted that, if desired, any data or analysis related to the user 336 or crowd of users 338 may be anonymized prior to being sent to one or more of the third-party networks 368, such as in accordance with privacy settings in user profiles (e.g., user profile 344 associated with the user 336, user profiles associated with respective users in the crowd of users 338, etc.).

The pandemic response module 352 is configured to execute processes based on receiving pandemic data from one or more of the third-party networks 368 via the third-party API module 350. The pandemic response module 352 may analyze such received information and provide notifications to the user 336 or crowd of users 338 including relevant information about the pandemic. The pandemic response module 352 may further collect and analyze physiological data of the user 336 or crowd of users 338 that may be relevant to the pandemic, and provides instructions to users who may be at risk due to the pandemic. Information about such at-risk users may also be provided to one or more of the third-party networks 368. The pandemic response module 352 may continually update the database 366 with relevant pandemic data including information about at-risk users. The pandemic response module 352, while described herein as processing information related to pandemics, may also be configured to process information related to epidemics and other outbreaks of diseases that do not necessarily reach the level of a pandemic. The pandemic response module 352 may also process information from the user 336 and crowd of users 338 so as to predict that a pandemic, epidemic or other disease outbreak is or is likely to occur. Thus, the functionality of the pandemic response module 352 is not limited solely to use in processing pandemic information.

The vital monitoring module 354 may monitor and analyze physiological data of the user 336 and crowd of users 338 to detect and mitigate pandemics, epidemics and other outbreaks or potential outbreaks of diseases. The physiological data may be analyzed to determine if there is evidence of a disease associated with a pandemic (e.g., shortness of breath associated with respiratory illness).

The location tracking module 356 is configured to track the location of user 336 and the crowd of users 338, to determine whether any of such users enter or exit regions associated with a pandemic or other outbreak of a disease. The location tracking module 356, in some embodiments, may alert users who have entered a geographic location or region associated with increased risk of exposure to an infectious disease (e.g., associated with an epidemic, pandemic or other outbreak). In some embodiments, various alerts, notifications and safety instructions are provided to the user 336 and crowd of users 338 based on their location. The threshold for detection of symptoms associated with an infectious disease (e.g., associated with an epidemic, pandemic or other outbreak) may be modified based on location of the user 336 and crowd of users 338. For example, the threshold for detecting a symptom (e.g., shortness of breath) may be lowered if the user 336 or crowd of users 338 are in high-risk locations for contracting an infectious disease.

The automated contact tracing module 358 is configured use the tracked location of the user 336 and crowd of users 338 (e.g., from the location tracking module 356) so as to determine possible contacts between such users, and also to assess risk of infection on a per-user basis. The automated contact tracing module 358 may also automate the delivery of notifications to the user 336 and crowd of users 338 based on potential exposure to other users or geographic regions associated with a pandemic or other outbreak of a disease. The automated contact tracing module 358 may further provide information regarding contacts between the user 336 and crowd of users 338 to one or more of the third-party networks 368 (e.g., indicating compliance with risk mitigation strategies for pandemic response).

The disease progression module 360 is configured to analyze physiologic data from the user 336 and crowd of users 338, and to determine whether such physiologic data is indicative of symptoms of a disease. As new physiologic data from the user 336 and crowd of users 338 is received, trends in such data may be used to identify the progression of a pandemic or other outbreak of a disease. The disease progression module 360 may be configured to monitor the progression of specific infectious diseases, such as infectious diseases associated with epidemics, pandemics or other outbreaks, based on any combination of: user indication of a contracted disease; one or more of the third-party networks 368 indicating that users have contracted a disease; the vital monitoring module 354 detecting a user contracting a disease with probability over some designated threshold; etc. The disease progression module 360 is further configured to compare disease progress for different ones of the users 336 and crowd of users 338 with typical disease progress to determine individual user health risk.

The in-home module 362 is configured to analyze location data from the user 336 and crowd of users 338, and to determine whether any of such users are in locations with stay-at-home or other types of quarantine, social distancing or other self-isolation orders or recommendations in effect. If so, the in-home module 362 may provide notifications or alerts to such users with instructions for complying with the stay-at-home, quarantine, social distancing or other self-isolation orders or recommendations, for mitigating an infectious disease, for preventing spread of the infectious disease, etc. The in-home module 362 may be further configured to provide in-home monitoring of infected patients that are quarantined or self-isolated at home, providing warnings to such users that leave the home, instructions for mitigating the disease, etc. The in-home module 362 may further provide in-home monitoring data to one or more of the third-party networks 368.

The essential workforce module 364 is configured to identify ones of the user 336 and crowd of users 338 that are considered part of an essential workforce or are otherwise considered essential personnel. Once identified, the essential workforce users' physiologic data may be analyzed to determine risk profiles for such users, and the algorithms implemented by modules 350 through 362 may be modified accordingly. As one example, the functionality of the in-home module 362 may be modified such that alerts or notifications are not sent to essential workforce users when leaving areas associated with stay-at-home, quarantine, social distancing or other self-isolation orders (e.g., those users would not receive alerts or notifications when traveling to or from their associated essential workplaces). Various other examples are possible, as will be described elsewhere herein.

Various ones of the pandemic response module 352, the vital monitoring module 354, the location tracking module 356, the automated contact tracing module 358, the disease progression module 360, the in-home module 362 and the essential workforce module 364 can further leverage the contextual and/or environmental data obtained from the accessory devices 315 in performing their various functionality.

As discussed above, physiological monitoring may benefit from additional contextual and/or environmental information about the conditions surrounding an individual under study (e.g., a subject, such as a human subject). For example, the value of a system that primarily acquires heart rate or core temperature data may be augmented by additional external sensing capability that targets exposure to infectious agents or insolation. This contextualization capability may, under some circumstances, need to be flexible, requiring different sensor modalities at different times with different individuals under study. In addition, some sensors may not be easily integrated into a single on-body monitoring device with a small form factor, and thus may need to be externalized to a different location on the same individual. These various modular devices require a dedicated BAN to manage their function and enable efficient data sharing.

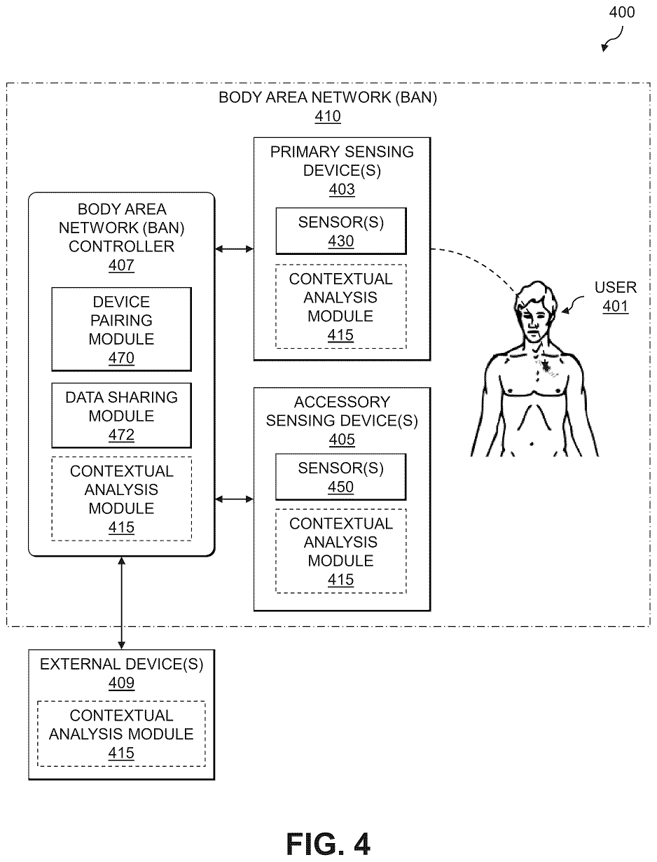

FIG. 4 shows aspects of a physiologic monitoring system 400 that is configured to management of multiple sensing devices in a BAN 410, including primary sensing devices 403 and accessory sensing devices 405. The primary sensing devices 403 in some embodiments are assumed to be relatively small form factor “on-body” sensing devices on a user or subject 401 (e.g., patch-module pairs as described elsewhere herein), with the accessory sensing devices 405 being relatively large form factor sensing devices, which may be “off-body” sensing devices. For example, the primary sensing devices 403 may include sensors 430 of a first type that can be used for physiologic monitoring on a patch interface or a module coupling with a patch interface as described elsewhere herein. The accessory sensing devices 405 may include sensors 450 of a second type which can be used for physiologic monitoring and/or for monitoring of a local environment of the BAN 410. More generally, the sensors 450 of the accessory sensing devices 405 are assumed to provide contextual and/or environmental information which can supplement physiologic monitoring data obtained using the sensors 430 of the primary sensing devices 403.

In some embodiment, the accessory sensing devices 405 comprise external sensor or accessory units that comprise one or more of the following, either singularly or in an array of multiple (potentially identical) devices: electrophysiological measuring devices, including but not limited to electrooculographs, electroglottographs, electrocardiographs, and electroencephalographs; optical sensors, including but not limited to ambient light sensors, spectrophotometers, closed-circuit television (CCTV), infrared and hyperspectral imagers; rangefinders and mapping devices, including but not limited to light detection and ranging (LIDAR), RADAR, and miniaturized opto-mechanical devices; sensors for body-exogenous and -endogenous biological agents and chemical compounds; dosimeters including but not limited to those configured for evaluating blast overpressure exposure, noise exposure, and radiation exposure; barometers; anemometers; accelerometers; gyroscopes; magnetometers; integrated transceivers for land navigation; audio transducers including speakers and microphones; dedicated machine learning devices for purposes including, but not limited to, sensor fusion, object identification, or threat early warning; and radio frequency transceivers generally.

The BAN 410 also includes a BAN controller 407 which is configured to perform management functions for the primary sensing devices 403 and the accessory sensing devices 405 which are part of the BAN 410. Such management functionality may include enabling a modular configuration of the primary sensing devices 403 and the accessory sensing devices 405, for flexible utilization of different ones of the primary sensing devices 403 and the accessory sensing devices 405 as needed for particular tasks. To do so, the BAN controller 407 implements a device pairing module 470 and a data sharing module 472. The device pairing module 470 provides functionality for pairing different ones of the primary sensing devices 403 and the accessory sensing devices 405 with the BAN 410 associated with the user or subject 401. The data sharing module 472 is configured to obtain and transmit data obtained from the sensors 430 of the primary sensing devices 403 and the sensors 450 of the accessory sensing device 405 for use in contextual analysis.

The contextual analysis is performed utilizing contextual analysis module 415 that may be implemented by the BAN controller 407, by one or more external devices 409. In the context of the system 200 of FIGS. 2A-2D, as an example, the BAN controller 407 may be implemented via the host device 230, with the external devices 409 comprising network-connected devices which are not part of a BAN formed between the sensing device 210, the accessory device 215, the stimulating device 220 and the host device 230. In the context of the system 300 of FIGS. 3A-3E, as an example, the BAN controller 407 may be implemented via the wireless gateway 340, with the external devices 409 comprising the AI wearable device network 348 and/or one or more of the third-party networks 368. In some cases, the contextual analysis logic 415 is also or alternatively implemented utilizing the primary sensing devices 403 and/or the accessory sensing devices 405. It should also be noted that, in some cases, the BAN controller 407 may be implemented by or as part of one or more of the primary sensing devices 403 and/or one or more of the accessory sensing devices 405.

The device pairing module 470 of the BAN controller 407 is configured to wirelessly pair the primary sensing devices 403 and the accessory sensing devices 405 in the BAN 410, such that the BAN controller 407 can serve as a network host for such devices. This may include, for example, pairing various external sensors and accessory units providing the accessory sensing devices 405, on demand as needed for particular tasks, with an existing BAN formed by the primary sensing devices 403. Consider, for example, a task that utilizes contextual and/or environmental information which cannot or is difficult to capture utilizing the sensors 430 of the primary sensing devices 403. In such a case, the contextual analysis module 415 may trigger the BAN controller 407 to utilize the device pairing module 470 to search for available accessory sensing devices 405 which are equipped with suitable sensors 450 for capturing the needed contextual and/or environmental information. If any suitable accessory sensing devices 405 are found, the device pairing module 470 will add such devices to the BAN 410. The data sharing module 472 can then obtain the needed contextual and/or environmental information from the sensors 450 of such accessory sensing devices 405, and then share such data with the contextual analysis module 415 (which, as discussed above, can be implemented in any combination of the primary sensing devices 403, the accessory sensing devices 405, the BAN controller 407 and the external devices 409). The device pairing module 470 and the data sharing module 472 may conduct device pairing activities and data transactions using various different networks and network types, including but not limited to UWB, Bluetooth, BLE, LoRA, Wifi, NFC, etc.

The entity implementing the contextual analysis module 415 may be referred to as a “remote receiver” that further processes both physiologic monitoring data (e.g., obtained from one or more of the sensors 430 of one or more of the primary sensing devices 403, and possibly from one or more the sensors 450 of one or more of the accessory sensing devices 405) as well as the contextual and/or environmental information (e.g., obtained from one or more of the sensors 450 of the accessory sensing devices 405). This may include receiving logistical data from different ones of the sensors 430 and/or the sensors 450 that are associated with the user or subject 401 under study, analyzing the logistical data to derive one or more parameters, and then taking some action.

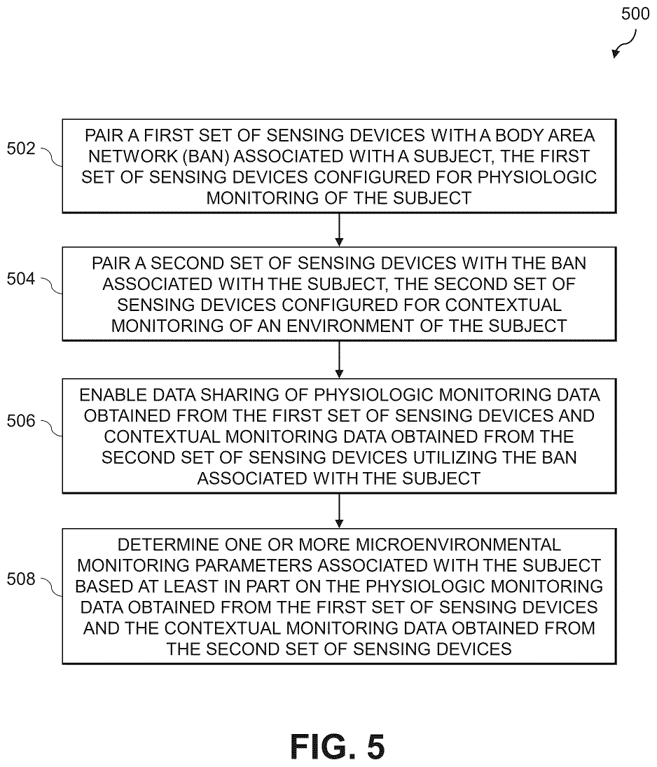

An exemplary process 500 for microenvironmental monitoring utilizing different types of sensing devices in a BAN associated with a subject will now be described with reference to the flow diagram of FIG. 5. It should be understood, however, that this particular process is only an example and that other types of processes for microenvironmental monitoring may be used in other embodiments as described elsewhere herein. The process 500 includes steps 502 through 508. The process 500 may be performed, for example by various devices that are in communication with sensing devices that are part of a BAN associated with a subject (including sensing and/or stimulating devices), such as a processing device that implements a BAN controller for a BAN associated with a subject.

In step 502, a first set of sensing devices are paired with the BAN associated with the subject, the first set of sensing devices configured for physiologic monitoring of the subject. A second set of sensing devices are paired with the BAN associated with the subject, the second set of sensing devices configured for contextual monitoring of an environment of the subject. Data sharing of physiologic monitoring data obtained from the first set of sensing devices and contextual monitoring data obtained from the second set of sensing devices is enabled in step 506 utilizing the BAN associated with the subject. One or more microenvironmental monitoring parameters associated with the subject are determined in step 508 based at least in part on the physiologic monitoring data obtained from the first set of sensing devices and the contextual monitoring data obtained from the second set of sensing devices.

The contextual monitoring data may comprise information characterizing exposure of the environment of the subject to at least one of: one or more infectious agents; insolation; radiation; blast overpressure; and noise.

At least one of the second set of sensing devices may comprise an electrophysiological measuring device, the electrophysiological measuring device comprising at least one of: an electrooculography sensor; an electroglottography sensor; and an electroencephalography sensor.

At least one of the second set of sensing devices may comprise an optical sensor device, the optical sensor device comprising at least one of: an ambient light sensor; a spectrophotometer sensor; a closed-circuit television sensor; an infrared sensor; and a hyperspectral imager sensor.

At least one of the second set of sensing devices may comprise a rangefinder and mapping device, the rangefinder and mapping device comprising at least one of: a light detection and ranging (LIDAR) sensor; a RADAR sensor; and a miniaturized opto-mechanical sensor.

At least one of the second set of sensing devices may comprise an electrophysiological measuring device, the electrophysiological measuring device comprising at least one of: an electrooculography sensor; an electroglottography sensor; and an electroencephalography sensor.

At least one of the second set of sensing devices may comprise a sensor for sensing at least one of body-exogenous and body-endogenous exposure to one or more biological agents and chemical compounds.

At least one of the second set of sensing devices may comprise a dosimeter configured to evaluate exposure to at least one of blast overpressure, noise and radiation.

At least one of the second set of sensing devices may comprise at least one of: a barometer; an anemometer; an accelerometer; a gyroscope; a magnetometer; an integrated transceiver for land navigation; and an audio transducer.

At least one of the second set of sensing devices may comprise a machine learning processing device configured for at least one of sensor fusion for sensor data from two or more different types of sensors, object identification, and threat early warning.

Enabling the data sharing of the physiologic monitoring data obtained from the first set of sensing devices and the contextual monitoring data obtained from the second set of sensing devices utilizing the BAN associated with the subject in step 506 may comprise utilizing at least one of ultrawideband (UWB) radio communications, Bluetooth radio communications, Bluetooth Low Energy (BLE) radio communications, long range (LoRa) radio communications, Wifi radio communications, and Near Field Communication (NFC) radio communications.

In some embodiments, step 504 comprises dynamically pairing respective ones of the second set of sensing devices with the BAN associated with the subject. Dynamically pairing a given one of the second set of sensing devices with the BAN associated with the subject may be performed responsive to identifying that a given one of the one or more microenvironmental monitoring parameters requires sensing data from a sensor type that is available on the given one of the second set of sensing devices but is not available from the first set of sensing devices.

The first set of sensing devices may comprise on-body sensing devices and the second set of sensing devices may comprise one or more off-body accessory devices.

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

What is claimed is:

1. An apparatus comprising:
at least one processing device comprising a processor coupled to a memory;
the at least one processing device implementing a body area network controller for a body area network associated with a subject, the at least one processing device being configured:
to pair a first set of sensing devices with the body area network associated with the subject, the first set of sensing devices configured for physiologic monitoring of the subject;
to pair a second set of sensing devices with the body area network associated with the subject, the second set of sensing devices configured for contextual monitoring of an environment of the subject;
to enable data sharing of physiologic monitoring data obtained from the first set of sensing devices and contextual monitoring data obtained from the second set of sensing devices utilizing the body area network associated with the subject; and
to determine, based at least in part on the physiologic monitoring data obtained from the first set of sensing devices and the contextual monitoring data obtained from the second set of sensing devices, one or more microenvironmental monitoring parameters associated with the subject.

2. The apparatus of claim 1, wherein the contextual monitoring data comprises information characterizing exposure of the environment of the subject to at least one of:
one or more infectious agents;
insolation;
radiation;
blast overpressure; and
noise.

3. The apparatus of claim 1, wherein at least one of the second set of sensing devices comprises an electrophysiological measuring device, the electrophysiological measuring device comprising at least one of:
an electrooculography sensor;
an electroglottography sensor; and
an electroencephalography sensor.

4. The apparatus of claim 1, wherein at least one of the second set of sensing devices comprises an optical sensor device, the optical sensor device comprising at least one of:
an ambient light sensor;
a spectrophotometer sensor;
a closed-circuit television sensor;
an infrared sensor; and
a hyperspectral imager sensor.

5. The apparatus of claim 1, wherein at least one of the second set of sensing devices comprises a rangefinder and mapping device, the rangefinder and mapping device comprising at least one of:
a light detection and ranging (LIDAR) sensor;
a RADAR sensor; and
a miniaturized opto-mechanical sensor.

6. The apparatus of claim 1, wherein at least one of the second set of sensing devices comprises an electrophysiological measuring device, the electrophysiological measuring device comprising at least one of:
an electrooculography sensor;
an electroglottography sensor; and
an electroencephalography sensor.

7. The apparatus of claim 1, wherein at least one of the second set of sensing devices comprises a sensor for sensing at least one of body-exogenous and body-endogenous exposure to one or more biological agents and chemical compounds.

8. The apparatus of claim 1, wherein at least one of the second set of sensing devices comprises a dosimeter configured to evaluate exposure to at least one of blast overpressure, noise and radiation.

9. The apparatus of claim 1, wherein at least one of the second set of sensing devices comprises at least one of:
a barometer;
an anemometer;
an accelerometer;
a gyroscope;
a magnetometer;
an integrated transceiver for land navigation; and
an audio transducer.

10. The apparatus of claim 1, wherein at least one of the second set of sensing devices comprises a machine learning processing device configured for at least one of sensor fusion for sensor data from two or more different types of sensors, object identification, and threat early warning.

11. The apparatus of claim 1, wherein enabling the data sharing of the physiologic monitoring data obtained from the first set of sensing devices and the contextual monitoring data obtained from the second set of sensing devices utilizing the body area network associated with the subject comprises utilizing at least one of ultrawideband radio communications, Bluetooth radio communications, Bluetooth Low Energy radio communications, long range (LoRa) radio communications, Wifi radio communications, and Near Field Communication (NFC) radio communications.

12. The apparatus of claim 1, wherein pairing the second set of sensing devices with the body area network associated with subject comprises dynamically pairing respective ones of the second set of sensing devices with the body area network associated with the subject.

13. The apparatus of claim 12, wherein dynamically pairing a given one of the second set of sensing devices with the body area network associated with the subject is performed responsive to identifying that a given one of the one or more microenvironmental monitoring parameters requires sensing data from a sensor type that is available on the given one of the second set of sensing devices but is not available from the first set of sensing devices.

14. The apparatus of claim 12, wherein the first set of sensing devices comprise on-body sensing devices and wherein the second set of sensing devices comprise one or more off-body accessory devices.

15. A method performed by at least one processing device implementing a body area network controller for a body area network associated with a subject, the method comprising:
pairing a first set of sensing devices with the body area network associated with the subject, the first set of sensing devices configured for physiologic monitoring of the subject;
pairing a second set of sensing devices with the body area network associated with the subject, the second set of sensing devices configured for contextual monitoring of an environment of the subject;
enabling data sharing of physiologic monitoring data obtained from the first set of sensing devices and contextual monitoring data obtained from the second set of sensing devices utilizing the body area network associated with the subject; and
determining, based at least in part on the physiologic monitoring data obtained from the first set of sensing devices and the contextual monitoring data obtained from the second set of sensing devices, one or more microenvironmental monitoring parameters associated with the subject.

16. The method of claim 15, wherein pairing the second set of sensing devices with the body area network associated with subject comprises dynamically pairing respective ones of the second set of sensing devices with the body area network associated with the subject.

17. The method of claim 16, wherein dynamically pairing a given one of the second set of sensing devices with the body area network associated with the subject is performed responsive to identifying that a given one of the one or more microenvironmental monitoring parameters requires sensing data from a sensor type that is available on the given one of the second set of sensing devices but is not available from the first set of sensing devices.

18. A computer program product comprising a non-transitory processor-readable storage medium having stored therein executable program code which, when executed, causes at least one processing device implementing a body area network controller for a body area network associated with a subject to perform steps of:
pairing a first set of sensing devices with the body area network associated with the subject, the first set of sensing devices configured for physiologic monitoring of the subject;
pairing a second set of sensing devices with the body area network associated with the subject, the second set of sensing devices configured for contextual monitoring of an environment of the subject;
enabling data sharing of physiologic monitoring data obtained from the first set of sensing devices and contextual monitoring data obtained from the second set of sensing devices utilizing the body area network associated with the subject; and
determining, based at least in part on the physiologic monitoring data obtained from the first set of sensing devices and the contextual monitoring data obtained from the second set of sensing devices, one or more microenvironmental monitoring parameters associated with the subject.

19. The computer program product of claim 18, wherein pairing the second set of sensing devices with the body area network associated with subject comprises dynamically pairing respective ones of the second set of sensing devices with the body area network associated with the subject.

20. The computer program product of claim 19, wherein dynamically pairing a given one of the second set of sensing devices with the body area network associated with the subject is performed responsive to identifying that a given one of the one or more microenvironmental monitoring parameters requires sensing data from a sensor type that is available on the given one of the second set of sensing devices but is not available from the first set of sensing devices.
